(19) United States
(12) Patent Application Publication
(10) Pub. No.: US 2026/0140107 A1
(43) Pub. Date: May 21, 2026

(54) IN-VITRO TISSUE MODEL AND TEST METHOD

(71) Applicant: Otsuka Medical Devices Co., Ltd., Tokyo (JP)

(72) Inventors: Liang ZHAI, Belmont, CA (US); Gexsy NAVA, East Palo Alto, CA (US)

(21) Appl. No.: 19/392,031

(22) Filed: Nov. 17, 2025

Related U.S. Application Data
(60) Provisional application No. 63/721,266, filed on Nov. 15, 2024.

(51) Int. Cl. G01N 33/50; A61B 17/00; A61B 18/00; A61B 18/14; A61B 18/18; A61B 18/28; A61N 7/00; A61N 7/02; C12N 5/077

(52) U.S. Cl. CPC G01N 33/5082; A61B 18/1492; A61B 18/1815; A61B 18/28; A61N 7/02; C12N 5/0652; G01N 33/5061; A61B 2017/00725; A61B 2018/0022; A61B 2018/00577; A61B 2018/00779; A61B 2018/00845; A61B 2018/00863; A61N 2007/003; C12N 2503/04

(57) ABSTRACT

An in-vitro tissue model for use with a test article includes a tissue sample that includes homogeneous tuna meat having an isotropic thermal response, wherein the tuna meat is characterized by a plurality of visually distinct color states, each color state corresponding to a respective temperature to which the tuna meat has been heated within a range of 43° C. to 60° C., thereby providing a visual indication of the thermal effect of the test article within said range.

### PRIORITY

This application claims the benefit of priority under 35 U.S.C. § 119(e) to U.S. Provisional Application No. 63/721,266, filed Nov. 15, 2024, which is incorporated herein by reference in its entirety to provide continuity of disclosure.

### TECHNICAL FIELD

This application relates generally to medical apparatuses and systems that deliver energy to target an anatomical location of a subject. More specifically, this application relates to an in-vitro tissue model, test kit, and test method used to test such apparatuses and systems.

### BACKGROUND

High blood pressure, also known as hypertension, commonly affects adults. Left untreated, hypertension can result in renal disease, arrhythmias, and heart failure. In recent years, the treatment of hypertension has focused on interventional approaches to inactivate the renal nerves surrounding a renal artery. Autonomic nerves tend to follow blood vessels to the organs that they innervate. Intraluminal devices, such as catheters, may reach specific structures, such as the renal nerves, which are proximate to the lumens in which the catheters travel. Accordingly, catheter-based systems can deliver energy from within the lumens to denervate the renal nerves in or in proximity to the vessel walls.

One approach to renal denervation uses radio frequency (RF) energy. The RF energy is delivered to a catheter having multiple electrodes placed against the intima of the renal artery to create an electrical field in the vessel wall and surrounding tissue. The electrical field results in resistive (ohmic) heating of the tissue to ablate the tissue and the renal nerve passing through that tissue at a depth of about 2 to 4 mm. To treat the renal nerves surrounding the renal arteries, the RF electrodes are repositioned numerous times around the inside of the renal artery, including in the distal branches of the renal artery in close proximity with the kidneys.

Many of the problems associated with RF systems are solved by a system having an ultrasound transducer that emits one or more therapeutic doses of unfocused ultrasound energy, e.g., 2 to 3 ablation per renal artery. The ultrasound transducer can be mounted at a distal end of catheter, and the unfocused ultrasound energy can heat tissue adjacent to a body lumen within which the catheter (and the transducer) is disposed. Such unfocused ultrasound energy may, for example, ablate target nerves surrounding the body lumen, e.g., targeting a depth of about 6 mm, without damaging non-target tissue such as the inner lining of the body lumen or unintended organs outside of the body lumen.

Bench models are used in medical device research and development. For example, bench models can be used to test the catheter-based systems used to deliver energy to denervate renal nerves. The bench models can include, for example, phantom tissue models. Energy can be delivered into the tissue models to verify, evaluate, and/or optimize system design and performance.

### SUMMARY

The present disclosure is defined in the independent claims. Further embodiments of the present disclosure are defined in the dependent claims.

An in-vitro tissue model for use with a test article includes a tissue sample that includes homogeneous tuna meat having an isotropic thermal response, wherein the tuna meat is characterized by a plurality of visually distinct color states, each color state corresponding to a respective temperature to which the tuna meat has been heated within a range of 43° C. to 60° C., thereby providing a visual indication of the thermal effect of the test article within said range. An in-vitro test kit for use with a test article having at least one energy emitter is provided herein. The in-vitro test kit includes a tissue holder and an in-vitro tissue model for use with the test article. The in-vitro tissue model includes a tissue sample that includes homogeneous tuna meat having an isotropic thermal response, wherein the tuna meat is characterized by a plurality of visually distinct color states, each color state corresponding to a respective temperature to which the tuna meat has been heated within a range of 43° C. to 60° C., thereby providing a visual indication of the thermal effect of the test article within said range, wherein the in-vitro tissue model is configured to be held by the tissue holder. The tissue holder is configured to maintain the in-vitro tissue model in a predetermined spatial relationship relative to the at least one energy emitter.

A method of testing is provided herein. The test method includes providing an in-vitro tissue model comprising a tissue sample that includes homogeneous tuna meat having an isotropic thermal response, placing a tissue treatment device in proximity to the tissue sample, delivering, by the tissue treatment device, ablation energy into the tissue sample to cause a change in color of the tuna meat, and characterizing a performance parameter of the tissue treatment device based on the change in color.

A medical ablation catheter system is provided herein. The medical ablation catheter system includes an energy emitter configured to deliver ablation energy and a controller operably coupled to the energy emitter, the controller having a set of operational parameters stored therein for controlling the delivery of ablation energy, wherein said set of operational parameters is determined by a method comprising: (a) delivering ablation energy from a test catheter to an in-vitro tissue model comprising homogeneous tuna meat having an isotropic thermal response; (b) observing a plurality of visually distinct color states in the tuna meat corresponding to a range of temperatures between 43° C. and 60° C.; and (c) selecting the set of operational parameters based on the observed color states to achieve a desired therapeutic effect.

The above summary does not include an exhaustive list of all aspects of the present disclosure. It is contemplated that the present disclosure includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

A better understanding of the features and advantages of the present disclosure may be obtained by reference to the following detailed description that sets forth illustrative embodiments, in which the principles of the disclosure are utilized, and the accompanying drawings.

FIG. 1 is a perspective view of a tissue treatment system, consistent with embodiments of the present disclosure.

FIG. 2 is a side view of selected components of the tissue treatment system of FIG. 1, consistent with embodiments of the present disclosure.

FIG. 3 is a side view of selected components of the tissue treatment system of FIG. 1, consistent with embodiments of the present disclosure.

FIG. 4 is a perspective view of selected components of the tissue treatment system of FIG. 1 inserted into a body lumen, consistent with embodiments of the present disclosure.

FIG. 5 is a side view of an in-vitro test kit, consistent with embodiments of the present disclosure.

FIG. 6 is a perspective view of a portion of a tissue holder, consistent with embodiments of the present disclosure.

FIG. 7 is a perspective view of a portion of a tissue holder, consistent with embodiments of the present disclosure.

FIG. 8 is a side view of in-vitro tissue models, consistent with embodiments of the present disclosure.

FIG. 9 is a perspective view of an in-vitro tissue model, consistent with embodiments of the present disclosure.

FIG. 10 is a flowchart of a test method, consistent with embodiments of the present disclosure.

FIG. 11 is an end view of a lumen of an in-vitro tissue model, consistent with embodiments of the present disclosure.

### DETAILED DESCRIPTION

In-vitro tissue models and test kits, and test methods for using the in-vitro tissue models and test kits to test systems that use thermal energy, e.g., unfocused ultrasound energy to treat tissue, are provided herein. In certain embodiments, the test methods are applied to acoustic-based tissue treatment transducers, apparatuses, systems, and portions thereof. The systems may be catheter-based. The systems may be delivered intraluminally (e.g., intravascularly) so as to place a transducer within a target anatomical region of the subject, for example, within a suitable body lumen such as a blood vessel. Once properly positioned within the target anatomical region, the transducer can be activated to deliver unfocused ultrasonic energy radially outward so as to suitably heat, and thus treat, tissue within the target anatomical region. The transducer or piezoelectric material can be activated at a frequency, duration, and energy level suitable for treating the ablation target, e.g., the targeted tissue. In one non-limiting example, unfocused ultrasonic energy generated by the transducer or piezoelectric material or radio frequency (RF) energy transmitted by the electrodes may target select nerve tissue of the subject, and may heat such tissue in such a manner as to neuromodulate (e.g., fully or partially ablate, necrose, or stimulate) the nerve tissue. In certain embodiments, microwave energy transmitted by an antenna may be delivered to treat tissue, e.g., ablate tissue comprising nerves.

Neuromodulating renal nerves may be used to treat various conditions, e.g., pulmonary hypertension, chronic kidney disease (CKD), cardiovascular disease, atrial fibrillation, arrhythmia, stroke, heart failure, end stage renal disease, myocardial infarction, anxiety, contrast nephropathy, diabetes, obesity, non-alcoholic fatty liver disease, digestive disease, pancreatic cancer, other cancers, tumors, pain, polycystic kidney disease, asthma, sepsis, rheumatoid arthritis, chronic obstructive pulmonary disease (COPD), post-traumatic stress disorder (PTSD), sleep apnea, anxiety, depression, metabolic disorder, and insulin resistance, etc. It should be appreciated that tissue treatment systems may be used to treat other nerves in and/or around a body lumen, e.g., the nerves in and/or around a renal artery, superior mesenteric artery, inferior mesenteric artery, femoral artery, pelvic artery, portal vein, hepatic artery, common hepatic artery, gastroduodenal artery, splenic artery, gastric artery, celiac trunk, pulmonary artery, pulmonary vein, aorta, vena cava, etc., e.g., sympathetic nerves of the hepatic plexus within a hepatic artery responsible for blood glucose levels important to treating diabetes, or any suitable tissue, e.g., heart tissue triggering an abnormal heart rhythm, and is not limited to use in treating (e.g., neuromodulating) renal nerve tissue. In another example, a tissue treatment system is used to ablate sympathetic nerves of the renal arteries and a hepatic artery to treat diabetes or other metabolic disorders. In certain embodiments, the tissue treatment system is used to treat an autoimmune and/or inflammatory condition, such as rheumatoid arthritis, sepsis, Crohn's disease, ulcerative colitis, and/or gastrointestinal motility disorders by neuromodulating sympathetic nerves within one or more of a splenic artery, celiac trunk, superior or inferior mesenteric artery. In certain embodiments, the tissue treatment system is used to ablate nerve fibers in the celiac ganglion and/or renal arteries to treat hypertension. In certain embodiments, the transducers are used to treat pain, such as pain associated with pancreatic cancer, by, e.g., neuromodulating nerves that innervate the pancreas. Ultrasound or RF energy may also be used to ablate nerves of both the pulmonary vein and the renal arteries to treat atrial fibrillation. In other examples, ultrasound or RF energy may additionally or alternatively be used to ablate nerves innervating a carotid body in order to treat hypertension and/or chronic kidney disease.

As described below, embodiments can include a test method for testing a tissue treatment system. The tissue treatment system may include an ultrasound-based tissue treatment catheter, used to deliver unfocused ultrasonic energy radially outwardly to treat tissue within a target anatomical region, such as the renal nerves within a renal artery. It will be appreciated, however, that the test method may be used to test alternative catheters or ablation devices. Additionally, the tissue treatment system or device tested with the test method may be used in other parts of the body than the renal artery, and/or to treat other conditions, such as to treat sympathetic nerves of the hepatic plexus within a hepatic artery, e.g., a common hepatic artery, and/or to ablate heart tissue to treat atrial fibrillation. Further, the test method may be utilized for testing medical devices other than catheters, such as devices used to treat a patient via minimally-invasive or traditional surgical approaches, and/or devices used to treat a patient via extracorporeal delivery of energy into the patient. Thus, reference in this document to the tissue treatment system as being part of a renal denervation system, or being used in treating, e.g., neuromodulating, renal nerve tissue using ultrasound energy, is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal” may indicate a first direction relative to a reference point, such as a user. Similarly, “proximal” may indicate a second direction relative to the reference point, opposite to the first direction. Such terms are provided to establish relative frames of reference, however, and are not intended to limit the use or orientation of in-vitro test models or test kits to a specific configuration described in the various embodiments below.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

Existing bench models used in medical device research and development lack the capability to consistently identify the ablation lesion boarders with high precision when visually assessing gross samples. The bench models use tissue to substitute for target human tissue to observe ablation effects. However, beef, pork, or chicken tissue models decolorize only after being denatured at a high thermal dose or temperature. More particularly, the tissue models change color when ablation technologies that apply a higher thermal dose or temperature are used, but experience slight or no visible color change at a lower thermal dose, such as that utilized by some ablation devices, e.g., ultrasound ablation devices. The existing tissue models therefore do not allow for reliable visualization of ablation lesions and their boundaries. Furthermore, existing bench model tissues tend to vary widely from sample to sample, e.g., when taken from different animals, and therefore the existing test methodologies require a large number of samples to generate statistically relevant results. Accordingly, there is a need for a bench model, e.g., an in-vitro tissue sample, which has good sensitivity, e.g., color change, at lower energies and temperatures of ablation, and which is reliable from sample to sample, to allow for reliable visualization of ablation lesions and their boundaries.

Lesion formation is highly sensitive to multiple, interdependent biophysical parameters. For example, computational models show that the relationship between acoustic attenuation and lesion depth is non-linear, where lesion extent can increase or decrease at higher attenuation values. Similarly, parameters such as tissue specific heat and thermal conductivity directly influence the final temperature achieved. Existing tissue models, due to their heterogeneity and high-temperature response thresholds, fail to provide a reliable physical means to study and validate these complex, sensitive interactions, especially for low-dose energy systems.

In an aspect, an in-vitro tissue model includes a tissue sample having a lumen for a catheter of a tissue treatment system to be located within during testing. The tissue sample can include tuna meat having a consistency that is temperature sensitive. More particularly, the thermal and acoustic properties, such as thermal capacity and thermal conduction, are such that a color of the tuna meat changes when the tissue sample is heated to a temperature at which thermal energy, e.g., unfocused ultrasound energy, radiofrequency energy or microwave energy, forms a lesion. For example, the temperature can be in a range of 53-57° C. Accordingly, the tissue model can be used to provide a reliable visualization of ablation lesions and their boundaries, for use in evaluating, verifying or optimizing engineering parameters and characterizing treatment outcomes, e.g., during product development or for regulatory submissions.

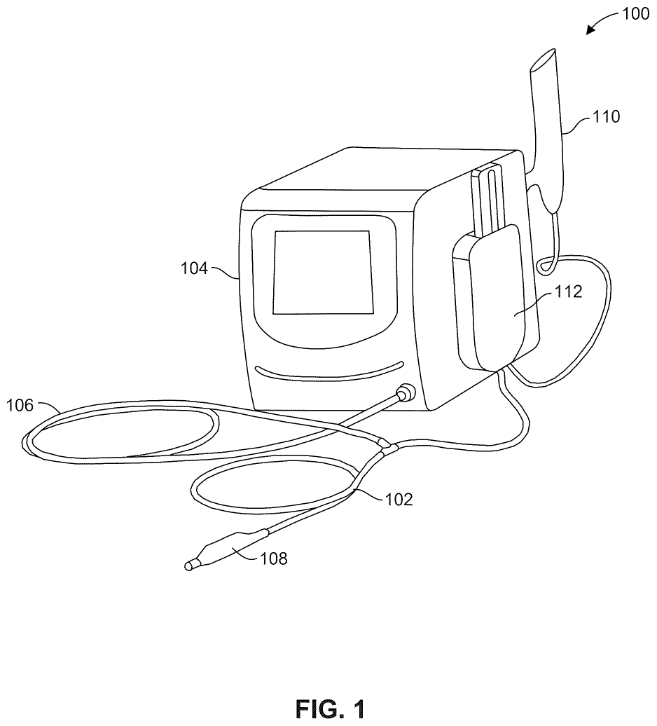

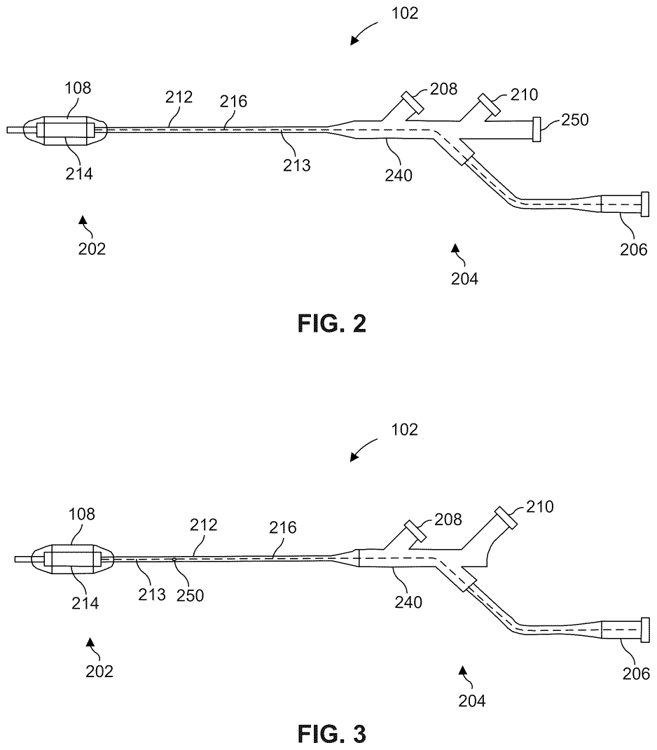

Referring to FIG. 1, a perspective view of a tissue treatment system is shown, consistent with embodiments of the present disclosure. An in-vitro tissue model or test kit, as described below, can be used to test a tissue treatment system 100. The tissue treatment system 100 may be a catheter-based system. More particularly, the tissue treatment system 100 can include a tissue treatment catheter 102 that can be delivered intraluminally, e.g., intravascularly, to a target anatomical region of a subject. When so placed, a transducer 214 of the tissue treatment system 100 (FIG. 2) can be positioned within a target anatomy, e.g., within a body lumen such as a blood vessel. The tissue treatment catheter 102 may include an energy emitter configured to emit therapeutic energy. The energy emitter may be one or more ultrasound transducers, one or more RF electrodes, one or more microwave emitters, or one or more emitters of other energy. As described below, the one or more energy emitters can be one or more ultrasound transducers 214 that may be disposed within a balloon 108. The transducer 214 can be activated to deliver unfocused ultrasonic energy radially outwardly so as to suitably heat, and thus treat, tissue within the target anatomical region. The transducer 214 can be activated at a frequency, time, and energy level suitable for treating the targeted tissue. According to other elements, the tissue treatment system 100 can include an energy delivery component that delivers RF energy, focused ultrasound, microwave energy, or other energy to tissue of the target anatomy. According to other embodiments, the tissue treatment system 100 may be a device other than a catheter, such as a surgical device used to treat a patient via minimally-invasive or traditional surgical approaches, or a device used to treat a patient via extracorporeal delivery of energy into the patient. As used in this document, when a tissue treatment system 100 is tested, it may be referred to as a test article.

In certain embodiments, the test method and in-vitro tissue model are specifically adapted for the evaluation of a radiofrequency (RF) ablation device. Such a device may comprise a catheter including one or more RF electrodes at its distal end. For testing, the distal end of the RF catheter is placed in proximity to the tuna meat tissue sample such that the one or more RF electrodes are in direct physical contact with a surface of the tissue sample. For example, the electrodes may be pressed against an inner wall of the lumen defined within the tissue sample to simulate intraluminal contact with a vessel wall.

Upon activation, the RF device delivers electrical current, which generates resistive (ohmic) heating directly within the tuna meat where contact is made. This heating causes the characteristic, temperature-dependent color change of the tuna meat. Even though RF ablation can generate high temperatures, the gradual color response of the tuna meat model provides a significant advantage over conventional tissue phantoms, which typically exhibit only a binary “cooked” or “uncooked” state. The tuna model allows for a high-resolution visualization of the thermal gradient and the precise boundaries of the resulting RF lesion. This enables accurate characterization of device performance, such as lesion depth and width, and provides reliable, repeatable results due to the homogeneous nature of the tuna meat. Accordingly, the present invention provides a robust platform for verifying and optimizing the performance of RF-based ablation systems. As used herein, a “desired therapeutic effect” refers to the creation of a thermal lesion of a predetermined size, shape, and depth sufficient to achieve a specific clinical goal, such as the neuromodulation, ablation, or necrosis of target tissue, while minimizing damage to non-target tissue. For example, in the context of renal denervation, a desired therapeutic effect may comprise the formation of a circumferentially continuous lesion having a length of approximately 5 mm along the renal artery, and a depth extending from approximately 1.0 mm to approximately 6.0 mm radially outward from the lumen wall. The ability of the in-vitro tissue model to provide a high-resolution visual profile of the lesion enables the precise selection and validation of operational parameters required to achieve such a therapeutic effect.

In other embodiments, the test method and in-vitro tissue model are specifically adapted for developing and validating a vessel-sparing microwave ablation protocol. This strategy aims to create a controlled therapeutic lesion at a target depth while actively protecting the endothelium and inner layers of the vessel wall. The test article for this application comprises a microwave ablation catheter that includes both a microwave antenna and an integrated cooling mechanism, such as a fluid-filled balloon or an open-irrigation system, configured to cool the surface of the lumen.

For this protocol, a high-power, short-duration energy delivery is used, for example, at a power level in the range of approximately 80 to 120 Watts for a duration of no longer than 60 seconds. The objective is to deliver sufficient energy to create a therapeutic lesion at a controlled depth, such as approximately 8.0 mm, while the integrated cooling mechanism simultaneously removes heat from the tissue immediately adjacent to the lumen.

The in-vitro tuna model is uniquely capable of providing direct, physical confirmation of this vessel-sparing effect. After the procedure, analysis of the sectioned tuna meat will reveal a distinct thermal gradient visualized by the color change. A less significant color change (e.g., remaining pinkish-red) will be observed at the lumen surface where the cooling was most effective, while a more intense color change (e.g., reddish-brown to white) will be visible at a subsurface depth where the microwave heating was dominant. This allows a user to precisely quantify the depth of the preserved tissue layer and optimize the critical balance between microwave power, duration, and cooling flow rate to achieve the desired therapeutic effect safely. The homogeneity of the tuna meat ensures these complex thermal gradient measurements are reliable and repeatable.

In yet another embodiment, the test method and in-vitro tissue model are uniquely suited for the evaluation of a laser ablation device, particularly one designed for an intimal-sparing effect. Such a device may comprise a fiber-optic catheter configured to deliver laser energy, for example, at a wavelength of approximately 1,064 nm. For testing, the distal tip of the fiber-optic catheter is placed within the lumen of the tuna meat tissue sample. Upon activation, for example at a power of approximately 10 Watts for 20 seconds, the catheter emits a divergent or conical beam of laser energy into the tissue.

A primary advantage of the tuna model in this context is its ability to physically visualize and validate the subsurface heating mechanism characteristic of a 1,064 nm laser. Due to the optical properties of this wavelength, the energy is minimally absorbed at the immediate surface of the lumen wall but penetrates deeper into the tuna meat before being absorbed and converted to heat. The model's gradual, temperature-dependent color change provides a direct, high-resolution visualization of this phenomenon. The most intense color change, indicating the highest temperature, appears at a depth within the tuna meat, while the surface of the lumen wall exhibits a less significant color change, physically demonstrating the preservation of the surface tissue. This allows for the precise measurement of lesion depths consistent with those observed in preclinical studies, such as a median depth of approximately 2.2 mm with a maximum observed depth of up to 3.6 mm.

Furthermore, the in-vitro tuna model serves as a critical tool for exploring and validating the full therapeutic potential of the laser device. While certain experimental conditions may yield a median lesion depth of 2.2 mm, the underlying physics of the 1,064 nm wavelength supports the potential for deeper energy penetration, capable of creating therapeutic effects at depths of 5 mm or more. The tuna model provides an invaluable platform for device developers to safely and systematically test this capability. By varying parameters such as power and duration, an engineer can use the model to precisely map the resulting deeper lesions, using the visual color boundaries to confirm that the maximum lesion depth remains within a variety of application-specific, predetermined safety margins, which may be 8 mm, 10 mm, or 12 mm. Accordingly, the in-vitro tuna model provides an essential tool not only for confirming performance under standard operating parameters but also for safely characterizing the device's entire therapeutic window, enabling the optimization of energy delivery protocols to achieve a desired therapeutic depth while respecting critical safety limits.

The tissue treatment system 100 may include the catheter 102, a controller 104, and a connection cable 106. The tissue treatment system 100 further includes a balloon 108, a reservoir 110, a cartridge 112, and a control mechanism, such as a handheld remote control. In certain embodiments, the controller 104 is connected to the catheter 102 through the cartridge 112 and the connection cable 106. In certain embodiments, the controller 104 interfaces with the cartridge 112 to provide cooling fluid to the catheter 102 for inflating and deflating the balloon 108. The controller 104 can also be referred to as the control unit.

In an embodiment, a catheter 102 can include a compliant balloon 108 configured to accommodate a range of target vessel sizes, and corresponding test sample lumens. The compliant balloon 108 can accommodate differences in vessel lumen diameter along the artery length and between left and right renal arteries. For example, the compliant balloon 108 may be configured to treat a blood vessel having a vessel lumen diameter between 3 to 9 mm in diameter. Thus, the compliant balloon 108 can mitigate the need to use several different balloon catheters per procedure. Accordingly, the balloon 108 can reduce procedure times and complexity. In certain embodiments, a noncompliant balloon may be used.

Referring to FIG. 2, a side view of selected components of the tissue treatment system of FIG. 1 is shown, consistent with embodiments of the present disclosure. The tissue treatment catheter 102 can include a distal region 202 and a proximal region 204. The catheter 102 may have a length that depends on a treatment application. For example, in certain embodiments suitable for, e.g., renal denervation through a femoral access delivery method, the catheter 102 can have a working length (measured from a distal tip of the catheter 102 to a proximal hub 240 of the catheter 102) of 80 to 90 cm, e.g., 85 cm, in the femoral access delivery method. In embodiments suitable for, e.g., renal denervation through a radial access delivery method, the catheter 102 can have a working length of a comparatively longer length. More particularly, the working length can be 150 to 160 cm, e.g., 155 cm. Furthermore, an overall length of the catheter 102 for such application, including a length of cabling extending to an electrical coupling 206, can be longer. More particularly, the cabling can have a length of about 305 cm from the proximal hub 240 to the electrical coupling 206.

The catheter 102 can have a profile that is suitable to accessing a renal artery through the femoral and radial access locations. For example, the catheter 102 may be 4 to 6 French in diameter, e.g., 5 French. The profile is facilitated in part by a catheter shaft 212 having an outer diameter in a range of 0.050 to 0.060 inch, e.g., 0.057 inch.

The distal region 202 of the tissue treatment system 100 may be a portion of the device that is advanced into a target anatomy, e.g., a target vessel having a vessel wall, to treat the target vessel. The distal region 202 can include the balloon 108 mounted on a catheter shaft 212. The balloon 108 can be a compliant balloon having the characteristics described in detail below. For example, the balloon 108 can have a cylindricity that supports and centers an energy emitter such as a transducer 214 within a range of vessel diameters, and thus, contributes to uniform energy delivery. In certain embodiments, a noncompliant balloon may be used.

The catheter shaft 212 can be an elongated tubular structure that extends longitudinally from a proximal end to a distal end. The balloon 108 can be mounted and supported on the catheter shaft 212 at the distal end. Furthermore, the ultrasound transducer 214 can be mounted at the distal end of the catheter shaft 212 and contained within an interior of the balloon 108. More particularly, the transducer 214 can be located in the interior. Accordingly, the catheter shaft 212 can facilitate delivery of a cooling fluid to the balloon 108 and delivery of electrical energy to the transducer 214.

The catheter shaft 212 can include one or more lumens that may be used as fluid conduits, electrical cabling passageways, guidewire lumens, or the like. In an embodiment, the catheter shaft 212 can include a guidewire lumen 213 that is shaped, sized, and otherwise configured to receive a guidewire. In an embodiment, the guidewire lumen 213 is an over-the-wire type guidewire lumen, extending from a distal tip of the catheter 102 through an entire length of the catheter shaft 212 to an exit port 250 in the proximal hub 240 of the catheter 102. As described below, the lumen(s) of the catheter shaft 212 may also communicate inflation/cooling fluid from the proximal region 204 to the balloon 108 during balloon expansion.

In an embodiment, a transducer 214 is mounted on the tissue treatment catheter 102 at the distal region 202, within the interior of the balloon 108. The transducer 214 can be an ultrasound transducer 214 used to emit energy toward the vessel wall. For example, the transducer 214 can emit ultrasound energy circumferentially, e.g., 360 degrees, around the vessel wall. In an embodiment, electric cabling 216 extends from the proximal region 204 to the distal region 202, and is connected to the transducer 214 to generate energy for emission to target tissue.

The ultrasound transducer 214 may include first and second electrodes that are arranged on either side of a cylindrical piezoelectric material, such as lead zirconate titanate (PZT). To energize the transducer 214, a voltage is applied across the first and the second electrodes at frequencies selected to cause the piezoelectric material to resonate, thereby generating vibration energy that is emitted radially outward from the transducer 214. The transducer 214 is designed to provide a uniform and predictable emission profile, to inhibit damage to surrounding non-target tissue. In addition, a cooling fluid is circulated through the balloon 108, both prior to, during, and after activation of the transducer 214, so as to reduce heating of an inner lining of the body lumen and to cool the transducer 214. In this manner, the peak temperatures achieved by tissue within the cooling zone remain lower than for tissue located outside the cooling zone. While computational models can predict this protective effect, their accuracy at the inner lesion boundary near the cooled surface is often limited. The tuna meat model overcomes this limitation by providing a direct, physical visualization of the true inner lesion boundary through its temperature-sensitive color change. This allows for a more accurate assessment of the efficacy of the cooling system and the safety margin at the tissue-lumen interface than is achievable through simulation alone.

The proximal region 204 may include one or more connectors or couplings. The connectors or couplings can be electrically connected to the transducer 214 via the electric cabling 216. For example, the proximal region 204 may include one or more electrical coupling 206 that connects to a proximal end of the electric cabling 216. A distal end of the electric cabling 216 can be connected to the transducer 214.

The catheter 102 may be coupled to the controller 104 by connecting the electrical coupling 206 to the connection cable 106. The connection cable 106 may be removably connected to the controller 104 or the catheter 102 via a port on the controller 104 or the catheter 102. Accordingly, the controller 104 can be used with several catheters 102 during a procedure by disconnecting the coupling of a first catheter, exchanging the first catheter with a second catheter, and connecting a coupling of the second catheter to the controller 104. In certain embodiments, e.g., where only one catheter needs to be used during a procedure, the connection cable 106 may be permanently connected to the controller 104.

In certain embodiments, the proximal region 204 of the catheter 102 may further include one or more fluidic ports. For example, the proximal hub 240 can include a fluidic inlet port 208 and a fluidic outlet port 210, via which an expandable member, e.g., the balloon 108, may be fluidly coupled to the reservoir 110 (FIG. 1). The catheter shaft 212 of the tissue treatment catheter 102 can include one or more fluid lumens fluidically coupled to the interior of the balloon 108. For example, the catheter shaft 212 can have a fluid lumen in fluid communication with the interior and extending to the fluidic inlet port 208 to receive an inflation/cooling fluid. Similarly, a fluid lumen can extend from the interior to the fluidic outlet port 210 to convey the inflation/cooling fluid away from the balloon 108. The reservoir 110 can therefore supply cooling fluid to the balloon 108 through the fluidic ports and fluid lumens. The reservoir 110 optionally may be included with the controller 104, e.g., attached to the outer housing of the controller 104 as shown in FIG. 1. Alternatively, the reservoir 110 may be provided separately.

In certain embodiments, a balloon model may be used to simulate clinical conditions in-vitro. The balloon model may involve embedding a renal artery (e.g., porcine) into a block of tuna meat. The renal artery is positioned within a bore formed in the tuna block, and the balloon is inflated inside the bore to simulate intraluminal energy delivery.

Proper contact between the balloon and the surrounding tissue is essential for consistent lesion formation. Inadequate bore sizing or improper balloon inflation may result in incomplete or malformed lesions, reducing the reliability of the test results.

A standardized procedural protocol is followed to ensure repeatability and precision. Standardized procedural protocol steps may include: thawing the tissue samples; cutting the tissue samples into specified dimensions; selecting bore size to match balloon size, allowing balloon to expand inside the renal artery and contact tissue; forming bore; inserting and inflating the balloon within the bore; performing sonification at body temperature (e.g., 37° C.); and conducting post-treatment analysis to assess lesion formation and temperature response.

In certain embodiments, conducting post-treatment analysis may include cutting the post-treatment tissue sample into at least two pieces; preparing tissue sample for analysis (e.g., removing water and other particulates); placing tissue sample under camera and performing imaging; and performing image analysis to assess lesion parameters. In certain embodiments, performing image analysis may include calculating image resolution; detecting a center point of the lesion; defining a seed of the lesion; detecting a lesion boundary; and calculating a lesion depth. As used herein, a seed may refer to an initial point or region within an image that serves as a starting location for an image processing algorithm.

Referring to FIG. 3, a side view of selected components of the tissue treatment system of FIG. 1 is shown, consistent with embodiments of the present disclosure. In an embodiment, the catheter 102 can have a rapid-exchange type guidewire lumen 213. More particularly, the guidewire lumen 213 can extend from the distal tip of the catheter 102 through a partial length of the catheter shaft 212 to an exit port 250 in the distal portion 202 of the catheter 102. For example, a distance from the distal tip to the rapid exchange port 250 may be in a range of 20 to 30 cm, e.g., 23 cm. The proximal hub 240 illustrated in FIG. 3 may differ from the proximal hub 240 illustrated in FIG. 2, given that the exit port 250 may be moved from the proximal portion 204 to the distal portion 202. Other components of rapid exchange version of the catheter 102 may be similar to those of the over-the-wire version of the catheter 102, and thus, the descriptions of the components illustrated in FIG. 2 can apply to similarly numbered components illustrated in FIG. 3.

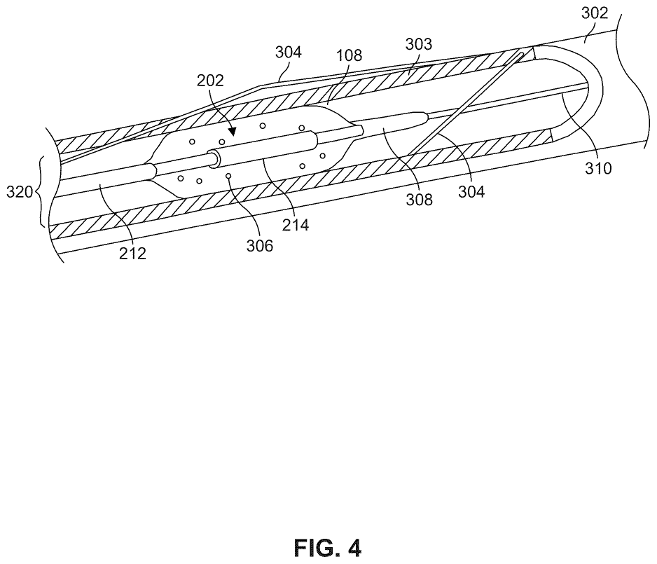

Referring to FIG. 4, a perspective view of selected components of the tissue treatment system of FIG. 1 inserted into a body lumen is shown, consistent with embodiments of the present disclosure. The tissue treatment system 100 can be inserted into a body lumen of a subject (or into a lumen of a tissue sample, as described below). For example, a distal region 202 of the catheter 102 of the tissue treatment system 100 can be advanced into a target vessel 302, e.g., a blood vessel such as a renal artery. The target vessel 302 can have a plurality of nerves 304 in an outer layer, e.g., an adventitia layer, of the target vessel 302. In an embodiment, the tissue treatment system 100 includes a guidewire support tip 308 having a lumen that connects to the guidewire lumen 213 of the catheter shaft 212. The support tip 308 can receive the guidewire 310 to allow the device to be tracked over a guidewire 310 to the target anatomy.

When the distal region 202 is disposed in the vessel lumen of the target vessel 302, the transducer 214 and the balloon 108 (or another suitable expandable member) are positioned radially inward from the plurality of nerves 304. The transducer 214 may be disposed partially or completely within the interior of the balloon 108. The balloon 108 can be filled with an inflation fluid 306, e.g., a cooling fluid, to expand the balloon 108. When the balloon 108 is inflated with the inflation fluid 306, the balloon 108 can contact an interior surface of a vessel wall 303, e.g., an intima, of the target vessel. The expanded balloon 108 may therefore have an inflated diameter equal to a lumen diameter 320 of the target vessel 302, and appose the target vessel 302 and center the transducer 214 within the target vessel 302.

In certain embodiments, the transducer 214 may be used to output an acoustic signal when the balloon 108 fully occludes the target lumen. The balloon 108 may center the transducer 214 within the target lumen. In certain embodiments, e.g., suitable for renal denervation, the balloon 108 may be a compliant balloon 108, as described below, which may be inflated in the patient during a procedure at a working pressure of about 1.4 to 2 atm using the inflation fluid 306. The balloon 108 is sized for insertion in the target lumen and, in the case of insertion of the renal artery, for example, the balloon 108 may be selected to have expansion sizes including outer diameters of one or more of 3.5 mm, 4.2 mm, 5 mm, 6 mm, 7 mm, 8 mm, or 9 mm. The balloon 108 may have a burst strength of greater than 45 psi.

In some embodiments, when inflated by being filled with the inflation fluid 306 under the control of the controller 104 within the target vessel 302, a balloon wall of the balloon 108 may be parallel with an outer surface of the transducer 214. Optionally, the balloon 108 may be inflated sufficiently as to be in apposition with the target vessel. For example, when inflated, the balloon 108 may at least partially contact, and thus be in apposition with, the inner wall of the target vessel. In other embodiments, the balloon 108 is configured not to contact the target vessel when expanded. The balloon 108 may be maintained at a specified size by pushing fluid into, e.g., via the inlet port 208, and pulling fluid out of, e.g., via the outlet port 210, the balloon 108 at a specified flow rate. More particularly, the inflation fluid 306 can circulate within the balloon 108 to expand the balloon 108.

In certain embodiments, the catheter 102 of the tissue treatment system 100 is balloonless and relies on blood flow to protect non-target tissue.

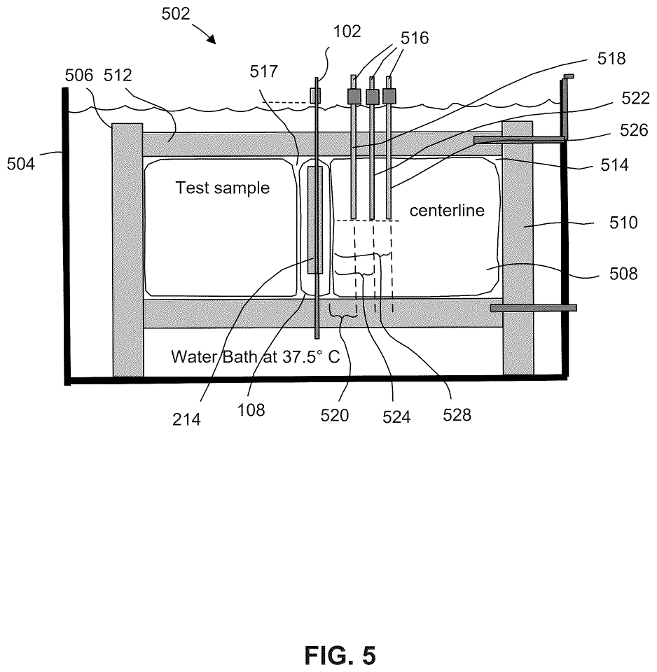

Referring to FIG. 5, a side view of an in-vitro test kit is shown, consistent with embodiments of the present disclosure. An in-vitro test kit 502 can provide a bench model for assessing the ablation of tissue and creation of lesions in the tissue by the tissue treatment catheter 102. The in-vitro test kit 502 can include a water bath 504, such as a water tank filled with water, and a tissue holder 506 submerged in the water bath 504. The tissue holder 506 can hold an in-vitro test model, as described in more detail below. More particularly, the in-vitro test model can be held in the tissue holder 506, and can include a tissue sample to simulate human tissue. The tissue holder 506 may hold the in-vitro test model whether a water bath 504 is used or not.

The water bath 504 can include the water tank filled with water. The water may be degassed water or tap water. The water may be heated to body temperature. For example, an immersion circulator may be placed in the water and activated to heat the water to 36-38° C., e.g., 37.5° C.

The tissue holder 506 can be submerged in the water bath 504. As described below, the tissue holder 506 can include a holder body 510 and a holder lid 512, and both components may be below a water line of the water in the tank. The holder lid 512 can be placed on the holder body 510 to enclose a holding cavity 514. For example, the tissue model 508 can be located in the holding cavity 514 and, thus, the tissue holder 506 can enclose the tissue model 508 under water during testing.

In some embodiments, the in-vitro test kit 502 may include the tissue holder 506, and omit the water bath 504. In such embodiments, the in-vitro test kit 502 may exclude a water bath 504. In such embodiments, the tissue treatment catheter 102 is coupled to the in-vitro tissue model in a different manner that provides for energy coupling between the tissue treatment catheter 102 and the in-vitro tissue model. As one example, where the tissue treatment catheter 102 includes one or more energy emitters configured as RF electrodes for ablation using RF energy, one or more of those RF electrodes may be placed in direct contact with a surface of the in-vitro tissue model, or placed within the lumen 517. As another example, where the tissue treatment catheter 102 is configured for ablation using ultrasound energy, one or more emitters of that ultrasound energy may be placed in direct contact with the in-vitro tissue model, or a coupling gel or other medium may be placed in contact with both the one or more emitters of that ultrasound energy and the in-vitro tissue model.

One or more temperature sensors 516 can be inserted into the tissue model 508 to sense a temperature of the tissue model 508 at various predetermined locations. In an embodiment, several temperature sensors 516 are located at respective radial distances from a lumen 517 of the tissue model 508. The temperature sensors 516 may, for example, include hypodermic thermocouple probes having a diameter of 0.2 mm (0.008 inch) and a length of 1 inch. The lumen 517 can be a channel extending through the tissue model 508 within which the balloon 108 of the tissue treatment catheter 102 can be disposed, for example. A first temperature sensor 518 can be a first radial distance 520 from the lumen 517, a second temperature sensor 522 can be a second radial distance 524 from the lumen 517, and a third temperature sensor 526 can be a third radial distance 528 from the lumen 517. In addition to being distanced from the lumen 517, the temperatures sensors can be spaced apart from the transducer 214, which is located within the balloon 108, by respective distances. The first distance 520, second distance 524, and third distance 528 may be progressively larger. Accordingly, as the transducer 214 sonicates and heats tissue in a radially outward direction, the first temperature sensor 518 can detect an increase in temperature nearest to the lumen 517, the third temperature sensor 526 can detect an increase in temperature farthest from the lumen 517, and the second temperature sensor 522 can detect an intermediate increase in temperature.

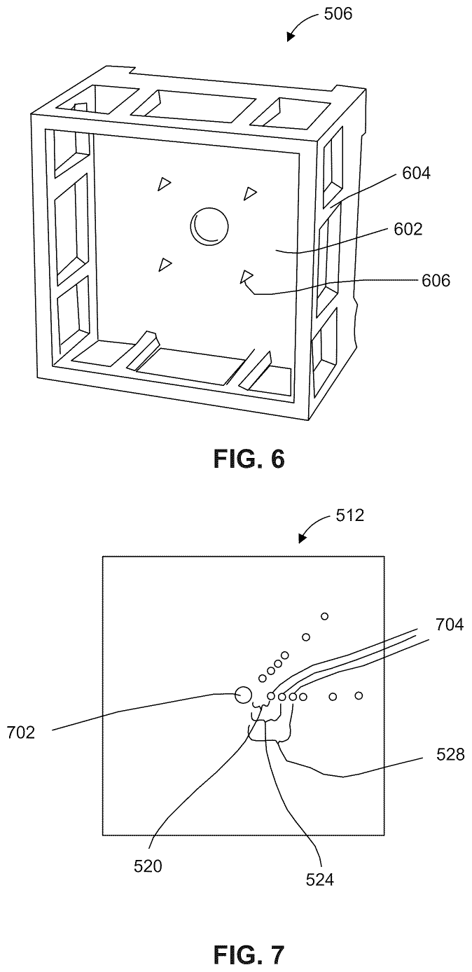

Referring to FIG. 6, a perspective view of a portion of a tissue holder is shown, consistent with embodiments of the present disclosure. The tissue holder 506 can includes a base 602 and a wall 604. The wall 604 may, for example, be a frame extending around the base 602. One or more cleats 606 may protrude upward from the base 602. The tissue model 508 can be loaded into the holding cavity 514 prior to testing, and may be engaged and steadied by the cleats 606. More particularly, the cleats 606 can include pointed prongs that engage the tissue and resist movement of the tissue relative to the base 602.

Referring to FIG. 7, a perspective view of a portion of a tissue holder is shown, consistent with embodiments of the present disclosure. The holder lid 512 can have a profile that matches a profile of the wall 604 of the tissue holder 506. For example, the wall 604 can extend along a rectangular path around the base 602, and the holder lid 512 can be rectangular. The lid 512 may therefore be placed on top of the wall 604 to cover the holding cavity 514. More particularly, when the lid 512 is placed on the wall 604, the holding cavity 514 can be contained between the base 602 and the lid 512.

The lid 512 can include one or more holes to allow passage of the tissue treatment catheter 102 or temperature sensors 516 through the lid into the holding cavity 514. In an embodiment, a central hole 702 is located along a central axis about which the holder lid 512 is distributed. The central hole 702 can be sized to allow a distal portion of the tissue treatment catheter 102, including the balloon 108 and the transducer 214, to pass through the lid. Several radial holes 704 can be located along radial lines extending outward from the central hole 702. For example, several radial holes 704 can be arranged along a same radial axis at respective distances from the central hole 702. A first radial hole 704 can be located at the first distance 520, a second radial hole 704 can be located at the second distance 524, and a third radial hole 704 can be located at the third distance 528 from the central hole 702. The radial holes 704 can allow the temperature sensors 516 to be inserted through the lid 512 into the tissue sample contained within the holding cavity 514. Accordingly, the lid 512 can guide the tissue treatment catheter 102 and the temperature sensors 516 to locate the components at predetermined positions within the tissue sample. The predetermined positioning of the components can ensure repeatable and reliable test data is produced from sample to sample.

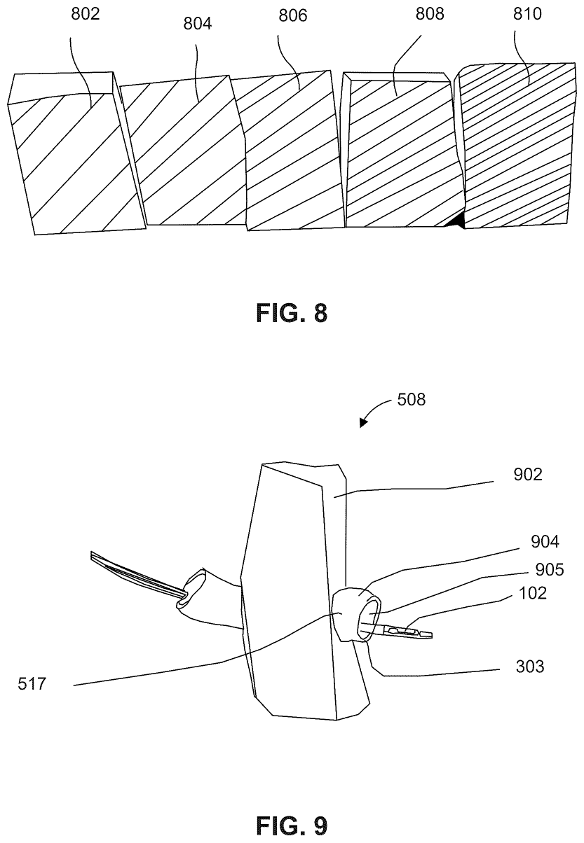

Referring to FIG. 8, a side view of an in-vitro tissue model is shown, consistent with embodiments of the present disclosure. The tissue holder 506 can contain the in-vitro tissue model 508, which may include a tissue sample having the lumen 517. The tissue sample can include tuna meat having a predetermined consistency. More particularly, as described below, the consistency of the tuna meat may be such that a color of the tuna meat changes in response to a temperature of the tissue sample.

Tuna meat provides a model for characterizing ablation outcome in human tissue because the thermal and acoustic properties of tuna meat, such as a heat capacity and heat conduction, can be similar to human muscle tissue. For example, a thermal capacity of the tuna meat can be in a range of 2700 J/g/° C. to 3600 J/g/° C., e.g., 3200 J/g/° C. For example, a thermal capacity of the tuna meat can be in a range of 2700 J/g/° C. to 3600 J/g/° C. Testing has confirmed that the properties of certain tuna meat samples align closely with nominal values used in validated predictive simulations, such as a specific heat of approximately 2372 J/Kg/K and a thermal conductivity of approximately 0.39 W/m/K. Furthermore, the tuna meat can have a thermal conductivity in a range of 0.3 W/m*K to 0.7 W/m*K, e.g., 0.5 W/m*K. Such tissue characteristics may be similar to the bulk characteristics of an in-vivo environment surrounding the vessel wall 303 of a renal artery. The tissue sample can therefore have such a thermal capacity, which accurately models the fat and muscle tissues of the in-vivo environment.

Advantageously, the tissue sample including the tuna meat can have an isotropic response to ultrasound ablation. More particularly, the tuna meat can be homogenous, and the response of the tuna meat to ultrasound can be isotropic. Such homogenous response can allow evaluation of circumferential uniformity in how the delivered acoustic energy distributes within the tissue surrounding the vessel wall 303. Furthermore, the homogeneity of the tuna meat may be absent in existing tissue models, such as those including chicken, pork, and beef tissue, which have more heterogenous tissue structures including different amounts of fat and connective tissues distributed unevenly in different directions. Accordingly, the tuna meat provides an unexpected benefit of responding uniformly in all directions to ablation energy and being insensitive to orientation of the transducer 214, in contrast to existing tissue models.

Furthermore, the tuna meat model provides a unique ability to physically validate the complex, non-linear physics of thermal ablation predicted by computational models. For example, sensitivity analyses show that lesion depth does not increase linearly with acoustic attenuation but instead peaks and then decreases. The gradual, temperature-dependent color change of the tuna meat allows for the direct physical observation and mapping of this non-linear effect across the lesion profile. This provides a crucial empirical confirmation that is impossible to achieve with conventional tissue models that exhibit only a binary high-temperature color change. Similarly, the model can be used to physically confirm the quasi-linear relationship between acoustic power and lesion depth, providing a reliable method for testing and calibrating device output.

As used herein, the term “homogeneous,” when used to describe the tuna meat tissue sample, refers to a material that exhibits a uniform composition and structural consistency throughout its volume, particularly with respect to the distribution of tissue types such as muscle, fat, and connective tissue. This homogeneity results in physical and acoustic, optical, or electrical properties that are consistent and predictable, leading to an “isotropic thermal response.” An isotropic thermal response means that when ablation energy is applied from a point or cylindrical source, the resulting thermal effects and lesion boundaries propagate uniformly in all directions. For the purpose of this disclosure, a tissue sample is considered homogeneous if its structural consistency is sufficient to allow for the formation of a circumferentially uniform lesion from a uniform energy source and to enable the reliable validation of device performance parameters, such as lesion depth and uniformity, without confounding effects from tissue variability. This is in contrast to heterogeneous tissues, such as pork or beef, which contain irregular distributions of different tissue types that lead to an anisotropic and unpredictable response to energy.

Advantageously, the tissue sample including the tuna meat can be used to characterize the lesion size in-vitro under a well-controlled or a constructed environment. The results of the in-vitro test are believed to more accurately and precisely corelate with the therapy effectiveness than currently available in-vitro tests. A bench test according to the current disclosure may be used to optimize therapy parameters (frequency, power, time, pulsing), dose and strategy for thermal ablation.

Another benefit of the tuna-based tissue model, over existing models, is the stiffness of the tuna meat, such as tuna muscle tissue. This inherent stiffness also enables the tissue sample to effectively function as a constraining structure for an expandable member, such as a balloon. Unlike softer tissues that might deform, stretch, or tear upon balloon inflation, the firm consistency of the tuna meat maintains the integrity of the bored lumen, ensuring the balloon expands into its intended cylindrical shape. This mechanical property is therefore essential for achieving the repeatable and reliable test conditions required for accurate device validation. Such stiffness can allow for the tissue sample to be cut open to measure an ablation lesion size after sonication is complete. The stiffness can correspond to a type of tuna meat. In an embodiment, the tuna meat is from a tuna of a species Thunnus albacares (yellowfin tuna). In certain embodiments, the tuna meat may include tuna muscle tissue. It has been shown that tuna meat of such type has the uniform temperature response described above and is stiff enough to support tissue sectioning during sample analysis.

As used herein, the term ‘stiffness’ refers to the mechanical property of the tuna meat that allows it to resist deformation, stretching, or tearing upon the application of force, such as from an inflating balloon. A tissue sample is considered to have sufficient stiffness if it can maintain the structural integrity of a bored lumen during the expansion of an expandable member therein, ensuring the expandable member achieves and maintains apposition with the bored lumen for repeatable and reliable test conditions. This property is in contrast to softer tissues that may deform and lead to malformed lesions.

Additional benefits of using tuna meat for bench models include its homogeneity, consistent thermal response, and predictable behavior under ablation conditions. These characteristics enable reliable visualization of lesion formation and temperature-dependent color changes, which are critical for evaluating treatment outcomes.

In contrast, other biological tissues, such as pork or liver, exhibit greater heterogeneity and variability in composition, including inconsistent tissue distribution and density. These factors reduce sensitivity to ablation and compromise repeatability across samples.

To better simulate clinical conditions, porcine renal arteries—despite their inherent heterogeneity—may be embedded within tuna blocks. This configuration allows for realistic modeling of anatomical structures while preserving the uniform thermal response of the surrounding tissue.

Synthetic gel models may also be used in some testing scenarios. However, these models often require higher temperatures (e.g., about 70° C.) to exhibit visible color changes and may suffer from uncertainty in material properties. As a result, gel models are less suitable for evaluating ablation techniques or for precise lesion boundary visualization.

Still referring to FIG. 8, several tissue samples are arranged in a row in the illustration. Each tissue sample can include a chunk of tuna meat cut to size from a larger tuna steak. For example, each tuna chunk can be a cube having sides of length 10-15 mm, e.g., 12 mm. As described below, the tissue sample can further include the lumen 517 bored through the tuna meat and, optionally, a vessel segment placed through the lumen 517 to simulate the vessel wall 303. The lumen 517 may be formed by boring the lumen 517 through the tuna meat 902. For example, a hole punch may be used. To achieve the necessary constraint on the balloon 108, the diameter of the bore created for the lumen 517 is selected to be substantially equal to or slightly smaller than the target inflated diameter of the balloon. For example, for a catheter intended for use in an 8 mm vessel, a bore of slightly less than 8 mm may be created to ensure a snug, interference fit upon balloon inflation. This ensures that the walls of the lumen 517 provide the structural support needed to prevent balloon deformation during testing.

The color of tuna meat can be temperature sensitive over a temperature range of interest. This provides a direct, visual indication of the thermal effect, which is a significant advantage over purely computational methods that rely on abstract calculated metrics for tissue necrosis, such as Cumulative Equivalent Minutes at 43° C. (CEM43C). By correlating specific colors to specific temperatures, the tuna model allows an observer to intuitively and accurately assess the extent of the effective thermal dose without complex post-processing, providing immediate and reliable feedback on lesion formation. For example, the tuna meat can change color over a range of 40-60° C. In an embodiment, the color of the tuna meat changes when the tissue sample is heated to a temperature in a range of 53-57° C. Notably, the color change may not be binary. More particularly, the color of the tuna can be a first color 802, as indicated by the cross-hatching, at a first temperature, e.g., 40° C., and the color can gradually change through a second color 804, a third color 806, and a fourth color 808, to reach a fifth color 810 at a fifth temperature, e.g., 60° C. The color can be proportional to the temperature, and can therefore change gradually based on the temperature change. The gradual color change, rather than a binary color change that can occur in existing tissue models 508, can allow an observer to determine, based on the color, the temperature of the ablated tissue. For example, when the tissue sample is heated to a temperature of 55° C., the tuna meat can have the fourth color 808, e.g., a reddish brown, which distinguishes from a deep red color of the first color 802 and a white color of the fifth color 810. The incremental increase in temperature can therefore be visually identified by the tuna meat color and heating of the tissue sample to the minimum temperature used to form a lesion with the tissue treatment catheter 102 can be determined.

Although the present disclosure refers to “color” as an indicator of thermal response in tissue samples, it should also be appreciated that other visual characteristics may also serve as effective indicators. These may include, but are not limited to, changes in hue, shade, saturation, brightness, or other perceptible variations in the appearance of the tissue. Such variations may be used to assess temperature-dependent effects and lesion formation, and may be quantified manually or through image analysis tools. Accordingly, references to “color” in this disclosure are intended to encompass a range of visual cures that reflect thermal response.

The measurement and control of tissue sample temperature is critical for accurate assessment of ablation effects. The tuna meat used in the in-vitro tissue model exhibits reliable and repeatable color changes at defined temperature thresholds, enabling visual quantification of thermal response. For example, color transitions may occur at specific temperature thresholds, such as approximately 41° C., 43° C., and 54° C., with each threshold corresponding to a distinct visual cue.

In an embodiment, the tuna meat can change color over a range of 40° C. to 55° C. The color of the tuna can be a first color 802 at a first temperature, e.g., at about 40° C., e.g., 40° C. to 42° C., and the color can gradually change to a second color 804, at a second temperature, e.g., at about 43° C., e.g., 43° C. to 45° C., a third color 806 at a third temperature, e.g., at about 46° C., e.g., 46° C. to 48° C., and a fourth color 808 at a fourth temperature, e.g., at about 49° C., e.g., 49° C. to 53° C., and reach a fifth color 810 at a fifth temperature, e.g., at about 54° C., e.g., 54° C. to 60° C., e.g., 54° C. to 55° C. In an embodiment, the energy delivery parameters, e.g., duration of ablation, power, frequency, or cooling fluid flow rate, used to reach the fifth color corresponding to a fifth temperature, e.g., about 54° C., e.g., 54° C. to 60° C., e.g., 54° C. to 55° C., can be programmed into the memory of the generator, such that an effective treatment can be performed with minimal damage to non-target tissue.

Prolonged exposure to temperatures above 43° C. may result in thermal denaturation or “cooking” of tissue, while exposure at approximately 54° C. can induce rapid ablation effects within seconds. Accordingly, treatment durations may be carefully monitored and controlled to ensure therapeutic efficiency while minimizing thermal injury to non-target tissue. In certain embodiments, treatment durations may be controlled to a defined range of time to avoid thermal injury, such as between 3 and 15 seconds. For example, treatment durations may be controlled to a duration of 3 seconds, 4 seconds, 5 seconds, 6 seconds, 7 seconds, 8seconds, 9 seconds, 10 seconds, 11 seconds, 12 seconds, 13 seconds, 14 seconds, or 15 seconds. In certain embodiments, treatment durations may be determined and controlled based on or as a function of temperature. For example, exposure at approximately 54° C. may be limited to shorter durations, such as 3 seconds, to avoid excess thermal damage, whereas exposure at lower temperatures may be controlled to longer durations.

As used herein, the term “about” refers to a degree of approximation that accounts for variations that may arise due to measurement limitations, experimental conditions, or manufacturing tolerances. When used in reference to a numerical value (e.g., temperature, concentration, dimension), “about” may encompass values that are within a reasonable range that would not materially affect the intended function or result. For example, when used in reference to temperature, the term “about” may refer to a tolerance of ±2° C. unless otherwise specified. Thus, “about 54° C.” may encompass a temperature range of 52° C. to 56° C. This range reflects the precision required for effective ablation while minimizing damage to non-target tissue. Unless otherwise specified, the range covered by “about” may further include deviations that a person of ordinary skill in the art would recognize as functionally equivalent in the context of the disclosure.

Referring to FIG. 9, a perspective view of an in-vitro tissue model is shown, consistent with embodiments of the present disclosure. The tissue model 508 can include tuna meat 902, as described above, having the lumen 517 extending longitudinally through the chunk of tuna meat. The lumen 517 may be sized to receive the balloon 108 having a predetermined size. More particularly, the lumen 517 may be sized to fit the balloon 108 of a test sample being tested. The lumen 517 may be 3 mm or great. In an embodiment, the lumen 517 has a diameter in a range of 3-8 mm, e.g., 5 mm. In some embodiments, the lumen 517 is not formed in the tuna meat 902. In some embodiments, the tuna meat 902 includes a lumen 517 defined therein, meaning that the lumen 517 may extend partially through, or entirely through, the tuna meat 902. In some embodiments, the tuna meat 902 includes a lumen 517 defined therethrough, meaning that the lumen 517 extends entirely through the tuna meat 902.

The lumen 517 may be formed by boring the lumen 517 through the tuna meat 902. For example, a hole punch may be used to form the lumen 517 in the tissue. In an embodiment, the hole punch includes a stainless steel tube. When the tube is pressed through the tuna meat 902, it can cut and remove a cylindrical portion of tuna meat 902. The remaining hole can form the lumen 517, and the tissue sample can have a bore cored through the middle.

In an embodiment, a tissue vessel is positioned in the lumen 517 to simulate the vessel wall 303. For example, the tissue model 508 can include a renal artery disposed within the lumen 517. The renal artery may be, for example, a porcine renal artery 904. The porcine renal artery 904 can have an arterial lumen 905 within which the tissue treatment catheter 102 can be placed during testing. More particularly, the balloon 108 of the tissue treatment catheter 102 can be expanded within the porcine renal artery 904 to contact the vessel wall 303 surrounding the arterial lumen 905 and to allow the transducer 214 to deliver ablation energy radially outward through the porcine renal artery 904 into the adjacent tuna meat 902. As described below, when the tuna meat 902 heats to a temperature consistent with temperatures created during unfocused ultrasound ablation procedures, the color of the tuna meat 902 will change to an identifiable, predetermined color that signals and provides high confidence that appropriate ablation has occurred.

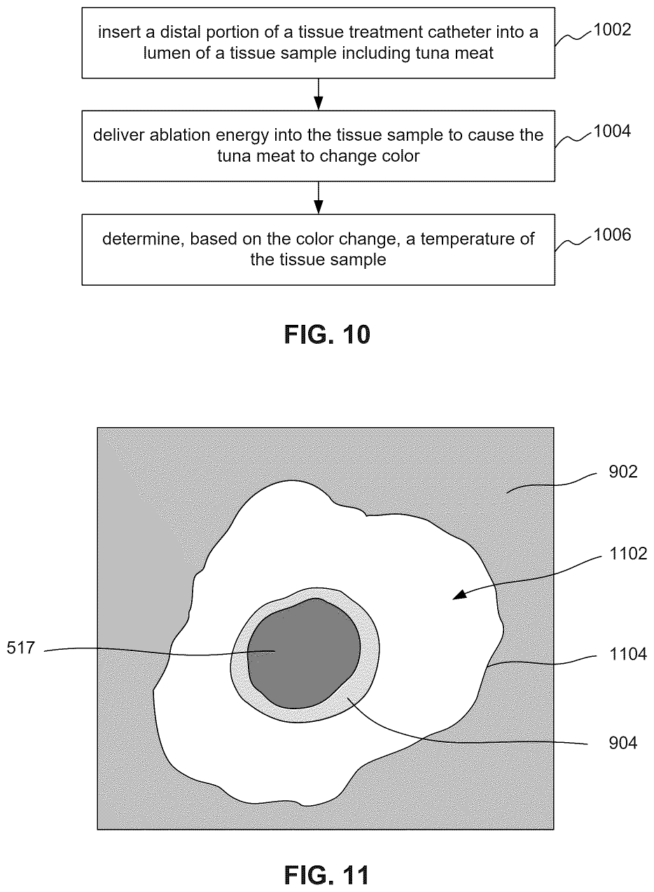

Referring to FIG. 10, a flowchart of a test method is shown, consistent with embodiments of the present disclosure. At operation 1002, a distal portion of the tissue treatment catheter 102 is inserted into the lumen 517 of the tissue sample. The tissue treatment catheter 102 may, for example, be threaded through a Tuohy-Borst adapter to deliver the balloon 108 downward through the holder lid 512 into the arterial lumen 905 inside of the lumen 517 of the tissue sample. Optionally, the balloon 108 may first be inflated into the renal artery and the subassembly of the distal catheter portion and the renal artery may then be placed into the lumen 517. In either case, the balloon 108 may extend through the lumen 517 and the transducer 214 may be aligned with the tuna meat 902 to allow generated ultrasonic waves to propagate radially outward into the tuna meat 902.

Alternatively, for test articles that include a non-compliant expandable balloon, the porcine renal artery may be omitted. In such embodiments, the non-compliant balloon is configured to be inflated so that it is in direct contact with an inner surface of the lumen defined by the tuna meat. This configuration is particularly suited for testing non-compliant balloons because their inflation to a predetermined, fixed diameter does not require apposition against a vessel wall to achieve a consistent shape. The inherent stiffness of the homogeneous tuna meat itself provides a reliable and constraining boundary for the non-compliant balloon, allowing for the direct assessment of energy delivery and lesion formation without the intermediary of a vessel segment.

The temperatures sensors 516 can be inserted into the tissue sample, e.g., through the radial holes 704, to locate the sensors at respective distances from the transducer 214. The temperatures sensors 516 can indicate when the tissue sample has reached body temperature and sonication can begin.

At operation 1004, ablation energy is delivered into the tissue sample to cause the tuna meat 902 to change color. For example, when testing a catheter-based ultrasound device, the energy can be delivered at specific power levels corresponding to the size of an expandable balloon, such as delivering approximately 27.9 Watts for a 3.5 mm balloon or approximately 38.0 Watts for an 8.0 mm balloon, for a predetermined duration such as 7 seconds. The sonication can heat the tissue sample, and the color of the tuna meat 902 can change accordingly. For example, as described above, when the tuna meat 902 reaches a temperature of 55° C., the tuna meat can have the fourth color 808, e.g., a characteristic reddish brown hue. More particularly, at operation 1006, the temperature of the tissue sample is determined based on the color change. The color change can be used to verify the temperature, as described further below. In some embodiments, the energy emitter(s) of the tissue treatment device may be placed into the water bath 504 in proximity to, and at a location spaced apart from, the tuna meat 902, and at operation 1004, the ablation energy is delivered into the tissue sample through the water of the water bath 504.

Referring to FIG. 11, an end view of a lumen of an in-vitro tissue model is shown, consistent with embodiments of the present disclosure. After sonicating the tissue sample, the sample can be sectioned. For example, the tissue sample may be placed in a cutting fixture and cut in half. The cut can reveal a generally circular lesion 1102 surrounding the lumen 517. The lesion 1102, which includes a cross-sectional area of tissue radially between the renal artery and an outer boundary 1104, can have the color that indicates the temperature that the tissue sample was heated to during ablation. More particularly, the color indicates the temperature that the tuna meat 902 was heated to between the renal artery (or lumen 517) and the outer boundary 1104.

The determined temperature may be confirmed by data collected by the temperature sensors 516. The temperature sensors 516 can be arranged at respective distances from the renal artery, as described above, and the sensing locations may align with the outer boundary 1104. For example, the outer boundary 1104 may be the second distance 524 from the lumen 517, and the second temperature sensor 522 may sense the temperature at that location. The sensed temperature data can confirm that the temperature is approximately 55° C., corresponding to the tuna meat 902 color.

The color of the tissue sample may indicate when the tissue is saturated and can no longer be ablated. In an embodiment, a change of color from a deep red (indicating unablated tuna meat 902) to white, in the tuna meat 902, can indicate that the tissue sample has reached the saturation point. When the tissue sample has been fully saturated, additional ablation may not be performed because the tissue is fully denatured.

As used herein, the term “predetermined safety margin” refers to a maximum allowable lesion dimension, such as a maximum lesion depth, which is established to ensure that therapeutic energy delivery does not cause unintended damage to non-target tissues or adjacent organs. The specific value of this margin is application-specific and is typically based on preclinical data, computational modeling, and anatomical considerations for a particular therapy. For example, in the context of certain renal denervation therapies, a predetermined safety margin for maximum lesion depth may be approximately 8.0 mm. For other therapeutic systems designed to treat different or larger anatomical structures, the predetermined safety margin may be larger, for example, approximately 10 mm or 12 mm. And for other therapeutic systems designed to treat different or smaller anatomical structures, the predetermined safety margin may be smaller, for example, approximately 6 mm, 4, or 2 mm.

The highly reliable and repeatable color change behavior of the in-vitro tuna meat model makes it particularly advantageous for verifying that a test article consistently creates lesions that remain within any such predetermined safety margin, regardless of its specific value, even when tested at the extremes of its operational tolerance. Identification of the outer boundary 1104 or color of the ablated tissue may be determined manually or automatically. For example, the sectioned tissue may be imaged by a camera, and the image data may be processed by one or more processors of an imaging system to detect the outer boundary 1104 and the color. More particularly, the one or more processors may use algorithms, e.g., artificial intelligence models, to analyze the image data and make determinations about the ablated tissue. For example, the detected color may be used to determine the temperature that the ablation zone was heated to during sonication.

While identification of the ablated tissue's color and outer boundary 1104 may be performed manually, in certain embodiments, the method utilizes an automated imaging system to ensure objectivity, precision, and repeatability. In such an automated embodiment, the system comprises a high-resolution imaging device, such as a digital camera, and one or more processors configured to execute an image analysis algorithm. This automated approach overcomes the inherent subjectivity of human visual assessment by quantifying the color of the ablated tissue into objective, numerical data. For example, the color of each pixel or region in an image of the sectioned tissue can be represented by values in a defined color space, such as RGB (Red, Green, Blue) or CIELAB (L*a*b*). These numerical color values are then correlated to specific temperatures using a predetermined calibration curve or lookup table stored in memory. The one or more processors can thereby generate a high-resolution thermal map of the lesion, allowing for the precise determination of the temperature achieved at any point within the tissue during sonication.

The tuna model described herein provides constant and well controlled test conditions to evaluate/test against specific system parameters, such as flow rate, total acoustic, optical, or electrical power output, duration, circumstantiality or acoustic intensity distribution etc. or combinations thereof. The tuna model can test various scenarios to mimicking nominal and/or worst case scenarios. The results can be used to validate theoretical or simulation results.

As used herein, “characterizing a performance parameter” of a tissue treatment device refers to the qualitative or quantitative assessment of any aspect of the device's function or its effect on the tissue model. Such characterization is not limited to a single measurement and may include, for example, measuring lesion dimensions such as depth and width; assessing the circumferential uniformity of energy delivery; correlating variations in energy delivery parameters (e.g., power, duration) with resulting lesion dimensions; and verifying non-linear biophysical relationships between tissue properties and lesion formation. In certain embodiments, lesion characterization may be performed using image analysis tools. These tools may include color analysis, which may encompass evaluating changes in hue, shade, saturation, brightness, or other perceptible visual cue, as indicators of thermal response. Image analysis may also include segmentation analysis or other automated image processing techniques to detect lesion boundaries or regions based on visual cues. For example, performing image analysis may include calculating image resolution; detecting a center point of the lesion; defining a seed of the lesion; performing image segmentation; performing color analysis; detecting a lesion boundary; and calculating a lesion depth. The use of image analysis tools is enhanced by high-resolution cameras (such as those integrated into mobile phones) to capture detailed images. These tools may be used to quantify color changes and lesion boundaries with greater precision than human observation alone, enhancing the accuracy and repeatability of post-treatment assessments.

The one or more processors may execute algorithms, such as machine learning models, to automatically perform image segmentation, which identifies the boundaries of the lesion with a precision superior to the human eye. For instance, an algorithm may be trained to detect the boundary between a first set of CIELAB values corresponding to unablated tissue and a second set of CIELAB values corresponding to ablated tissue at a specific temperature threshold. This process may involve defining a seed region within the lesion and using a region-growing or edge-detection algorithm to map the full extent of the color change. Once the lesion boundary is identified, the system can automatically calculate critical performance parameters, including maximum lesion depth, minimum lesion depth, average lesion depth, and lesion area, with a precision and repeatability that is superior to manual measurement.

Statistical analysis of lesion depth across multiple samples has demonstrated a generally linear relationship between energy delivery power and lesion depth. For example, lesion depth may increase proportionally with power settings, with observed changes in the 21-22 mm range under controlled conditions. Lesion depth may be measured using a maximum lesion depth, a minimum lesion depth, or an average lesion depth. Tolerance and variability are managed by analyzing groups of samples, allowing for robust characterization of treatment effects.

Technical reviews of experimental data confirm that the use of tuna meat as a tissue model provides more uniform energy distribution and consistent color changes behavior. These characteristics align closely with expected treatment temperatures and support the reliability of the model for evaluating ablation performance.

Additional considerations may influence the consistency and reliability of in-vitro testing outcomes. For example, the brand or the source of tuna meat used in the tissue model can affect performance. For instance, tuna meat sourced from the brand Natural Blue Wild Caught Ahi Tuna Steaks has demonstrated a more consistent thermal and acoustic properties than tuna meat sourced from other brands, which has resulted in more reliable color change and lesion formation.

Freezing and thawing procedures may also impact tissue handling and ablation results. For example, improper thawing may alter the structural integrity or thermal response of the tuna meat, leading to variability in lesion characterization. Standardized protocols for freezing and thawing are recommended to maintain consistency across test samples.

The use of balloon models in bench testing provides a controlled and repeatable environment for evaluating ablation systems. These models reduce reliance on animal studies by enabling efficient simulation of clinical conditions, thereby supporting ethical and cost-effective device development.

It will be appreciated that the tissue sample, test kit, and test method described above can have particularly advantageous application in low dose (low temperature) ablation regimes, when the tissue is ablated or necrosed without excessive loss of water. Such low dose regimes include, and are not limited to, those achieved with low-dose unfocused ultrasound. More particularly, whereas ablation using typical ablation electrodes can be used to heat tissue to 80-90° C. (high dose) to create a thermal lesion, temperatures achieved by unfocused ultrasound ablation to create a thermal lesion can be below 60° C. At the boundary of the thermal lesion, the temperature and thermal dose is lower but just sufficient to necrose the tissue. Existing tissue models 508 may not visually change under the low dose conditions. Tuna meat 902, on the other hand, does. Thus, the temperature of the ablation, and the extent of the thermal lesion, can be evaluated by visual inspection of the tuna meat 902. Accordingly, the tissue sample, test kit, and test method described above can be used to evaluate low dose ablation.

### EXPERIMENTAL EXAMPLES

To demonstrate the utility and unexpected advantages of the in-vitro tissue model, a series of experiments were conducted. The experiments were designed to (1) establish a baseline for lesion characterization using the tuna meat model, and (2) validate the model's sensitivity and reliability in assessing ablation system performance across a range of variable parameters, using data consistent with validated computational and theoretical models.

### General Experimental Setup

An in-vitro test kit was assembled. The tissue model comprised a block of tuna meat sourced from the brand Natural Blue Wild Caught Ahi Tuna Steaks, selected for its consistent thermal and acoustic properties. A lumen with a diameter between 3 mm and 8 mm was bored through the center of the tuna block. The block was then secured in a tissue holder and submerged in a water bath heated and maintained at a body temperature of approximately 37° C. A catheter-based ultrasound ablation device, serving as the test article, was inserted into the lumen. A plurality of hypodermic thermocouple probes were inserted into the tuna meat at predetermined radial distances from the lumen to record temperature data.

### Example 1: Baseline Lesion Characterization and Validation

A baseline test was conducted to characterize lesion formation under nominal conditions. A test article with an 8.0 mm diameter expandable balloon was used. The tissue model was prepared from a tuna meat sample exhibiting properties aligned with nominal simulation values, including an acoustic attenuation of approximately 0.5 dB/cm/MHz and a specific heat of approximately 2372 J/Kg/K.

The test article was activated to deliver a nominal acoustic power of 38.0 Watts for a sonication time of 7 seconds. Following sonication and a 10-second post-cool period, the tuna block was removed and sectioned for analysis. Visual inspection of the color change revealed a well-defined, circumferentially uniform lesion with a distal extent of approximately 5.7 mm. This result physically confirmed the predictions of a validated lesion extent simulator and corresponded to a thermal dose sufficient for tissue necrosis (equivalent to a CEM43C value of 240 minutes), demonstrating the model's accuracy under standard operating conditions.

### Example 2: Sensitivity Analysis for System Performance Verification

A series of tests were performed to demonstrate the tuna meat model's unique capability to physically verify system performance against known biophysical sensitivities.
A. Acoustic Attenuation Sensitivity: Tuna meat samples representing a range of acoustic attenuation values from 0.3 dB/cm/MHz to 1.5 dB/cm/MHz were tested at a constant power. The physical lesions formed in the samples demonstrated a non-linear relationship between attenuation and lesion depth. A peak lesion depth of approximately 6.34 mm was observed in the sample corresponding to an attenuation of 0.8 dB/cm/MHz, representing a 6% increase over the nominal 6.0 mm reference lesion. This result physically validated the complex, non-linear effect predicted by simulations, which cannot be observed in conventional tissue models.
B. Heat Capacity Sensitivity: Tuna meat samples representing a range of specific heat values from 2400 J/Kg−K to 4000 J/Kg−K were tested. A monotonic decrease in outer lesion extent was observed with increasing specific heat, confirming theoretical predictions. For an 8.0 mm balloon, the total change in inner lesion extent over this entire range was only 0.25 mm. This test confirmed that using tuna meat with a nominal specific heat of 2372 J/Kg/K provides a conservative, “worst-case” assessment for maximum lesion depth, a critical factor for safety analysis.
C. Acoustic Power Sensitivity: The test article's power was varied across its tolerance range of ±14% from the nominal power setting. The resulting lesion depths in the tuna model showed a quasi-linear relationship with input power, with lesion extents ranging from approximately 4 mm to 10 mm depending on balloon size and power level. This allowed for robust characterization of the system's performance envelope and confirmed that even at maximum power tolerance, the resulting lesion depth remained within a clinically acceptable safety limit of 8.0 mm.
D. Circumferential Uniformity Verification: A test article with a known, imperfect energy distribution (corresponding to a Uniformity Ratio (UR) of 0.42) was tested. The homogenous and isotropic nature of the tuna meat model allowed for precise 360-degree sectioning and visual analysis of the entire lesion boundary. The measured lesion was non-symmetrical, with a deeper extent in the sector of concentrated energy, yet the maximum depth was confirmed to be within the 8.0 mm safety requirement. This demonstrated the model's unique suitability for validating the circumferential uniformity of energy delivery, a critical parameter for cylindrical transducers.

The results of these experiments confirm that the in-vitro tuna meat model is not merely a tissue substitute, but a highly effective and sensitive diagnostic tool. It provides a reliable, repeatable, and visually intuitive method for characterizing ablation performance and validating complex system dynamics that are otherwise only predictable through abstract simulation.

Embodiments of an in-vitro tissue model, test kit, and test method are described above. More particularly, embodiments of the in-vitro tissue model, test kit, and test method are described, either explicitly or implicitly. The following paragraphs summarize some of the described embodiments. More particularly, embodiments are described in the following enumerated examples.
Example 1. An in-vitro tissue model for use with a test article, comprising a tissue sample that includes tuna meat having a consistency such that a color of the tuna meat changes when the tissue sample is heated by the test article to a temperature in a range of 53-57° C.
Example 2. The in-vitro tissue model of example 1, wherein the tissue sample includes a lumen defined therein.
Example 3. The in-vitro tissue model of example 1, wherein the tissue sample includes a lumen defined therethrough.
Example 4. The in-vitro tissue model of any of examples 2 through 3, further comprising a porcine renal artery disposed within the lumen.
Example 5. The in-vitro tissue model of any of examples 2 through 4, wherein the lumen has a diameter in a range of 3-8 mm.
Example 6. The in-vitro tissue model of any of examples 2 through 5, wherein the lumen is sized to receive at least a portion of the test article that emits energy.
Example 7. The in-vitro tissue model of any of examples 1 through 6, wherein the test article is configured to emit ultrasound energy, and wherein the tissue sample has an isotropic response to ultrasound energy.
Example 8. The in-vitro tissue model of any of examples 1 through 4, wherein the tissue sample has a thermal capacity in a range of 2700 kJ/kg/° C. to 3600 kJ/kg/° C.
Example 9. The in-vitro tissue model of any of examples 1 through 5, wherein the color is proportional to the temperature.
Example 10. The in-vitro tissue model of any of examples 1 through 6, wherein the tuna meat is from a tuna of a species Thunnus albacares.
Example 11. An in-vitro test kit for use with a test article having at least one energy emitter, comprising a tissue holder, and an in-vitro tissue model held by the tissue holder. The in-vitro tissue model includes a tissue sample having a lumen defined therein. The tissue sample includes tuna meat having a consistency such that a color of the tuna meat changes when the tissue sample is heated by the energy emitter to a temperature in a range of 43-60° C.
Example 12. The in-vitro test kit of example 11, wherein the in-vitro tissue model includes a porcine renal artery disposed within the lumen.
Example 13. The in-vitro test kit of any of examples 11 through 12, wherein the lumen has a diameter in a range of 3-8 mm.
Example 14. The in-vitro test kit of any of examples 11 through 13, wherein the tissue sample has a thermal capacity in a range of 2700 kJ/kg/° C. to 3600 kJ/kg/° C.
Example 15. The in-vitro test kit of any of examples 11 through 14, wherein the tuna meat is from a tuna of a species Thunnus albacares.
Example 16. The in-vitro test kit of any of examples 11 through 15, further comprising a water bath, wherein the tissue holder is configured to be submerged in the water bath.
Example 17. The in-vitro test kit of example 16, wherein the water bath is heated to body temperature.
Example 18. The in-vitro test kit of any of examples 16 through 17, wherein the at least one energy emitter is spaced apart from the in-vitro tissue model by water.
Example 19. The in-vitro test kit of any of examples 11 through 18, further comprising a plurality of temperature sensors located at respective radial distances from the lumen.
Example 20. A test method, comprising providing a tissue sample including tuna meat, and placing a tissue treatment device in proximity to the tissue sample. The test method includes delivering, by the tissue treatment device, ablation energy into the tissue sample to cause a color of the tuna meat to change. The test method includes determining, based on the color, a temperature of the tissue sample.
Example 21. The test method of example 20, wherein the tissue sample has a thermal capacity in a range of 2700 kJ/kg/° C. to 3600 kJ/kg/° C.
Example 22. The test method of any of examples 20 through 21, wherein the tuna meat is from a tuna of a species Thunnus albacares.
Example 23. The test method of any of examples 20 through 22, wherein the tissue treatment device is a catheter, further comprising inserting a distal portion of the tissue treatment catheter into a lumen defined in the tissue sample.
Example 24. The test method of example 23, wherein a porcine renal artery is disposed within the lumen.
Example 25. The test method of any of examples 23 through 24, wherein the lumen has a diameter in a range of 3-8 mm.
Example 26. The test method of any of examples 20 through 25, wherein the tissue treatment catheter includes a catheter shaft having a fluid lumen, a balloon mounted on the catheter shaft and having an interior in fluid communication with the fluid lumen, and an ultrasound transducer located in the interior.
Example 27. The test method of example 26, wherein the ablation energy is ultrasound energy.
Example 28. The test method of any of examples 20 through 27, wherein the tissue treatment device includes an ultrasound transducer; further comprising placing the tissue treatment device and the tissue sample in a water bath.
Example 29. The test method of any of examples 20 through 25, wherein the ablation energy is RF energy.
Example 30. The test method of any of examples 20 through 25 and 29, wherein the tissue treatment device comprises at least one RF electrode; wherein the placing a tissue treatment device in proximity to the tissue sample comprises contacting a surface of the tissue sample with the at least one RF electrode.
Example 31. An in-vitro tissue model for use with a test article, comprising:
a tissue sample that includes tuna meat having a consistency such that a color of the tuna meat changes to a first color when the tissue sample is heated by the test article to a first temperature, the color of the tuna meat changes to a second color when the tissue sample is heated by the test article to a second temperature, the color of the tuna meat changes to a third color when the tissue sample is heated by the test article to a third temperature, and the color of the tuna meat changes to a fourth color when the tissue sample is heated by the test article to a fourth temperature.
Example 32. The in-vitro tissue model of claim 31, wherein the first temperature is about 41° C.
Example 33. The in-vitro tissue model of any one of claims 31 to 32, wherein the second temperature is about 43° C.
Example 34. The in-vitro tissue model of any one of claims 31 to 33, wherein the third temperature is about 46° C.
Example 35. The in-vitro tissue model of any one of claims 31 to 34, wherein the fourth temperature is about 49° C.
Example 36. The in-vitro tissue model of any one of claims 31 to 35, wherein the color of the tuna meat changes to a fifth color when the tissue sample is heated by the test article to a fifth temperature.
Example 37. The in-vitro tissue model of claim 36, wherein the fifth temperature is about 54° C.
Example 38. A method for obtaining a response of a tissue model system to set of energy delivery parameters comprising: providing a tissue sample including tuna meat, placing a tissue treatment device in proximity to the tissue sample, delivering, by the tissue treatment device, ablation energy into the tissue sample to cause a color of the tuna meat to change color one or more times in response to a change in temperature between 41° C. and 54° C.
Example 39. An in-vitro tissue model for use with a test article, comprising: a tissue sample that includes tuna meat having a consistency such that the tuna meat undergoes a change color one or more times in response to a change in temperature between 41° C. and 54° C.
Example 40. An in-vitro tissue model for use with a test article, comprising: a tissue sample that includes tuna meat having a consistency such that the tuna meat undergoes a change color a plurality of times in response to a change in temperature between 41° C. and 54° C.
Example 41. An in-vitro tissue model for use with a test article, comprising: a tissue sample that includes tuna meat, and wherein a color of the tuna meat changes in response to the tissue sample being heated by the test article to a temperature in a range of 43-60° C.
Example 42. An in-vitro test kit for use with a test article having at least one energy emitter, comprising: a tissue holder; and an in-vitro tissue model held by the tissue holder, wherein the in-vitro tissue model includes a tissue sample having a lumen defined therein, wherein the tissue sample includes tuna meat, and wherein a color of the tuna meat changes when the tissue sample is heated by the at least one energy emitter to a temperature in a range of 43-60° C.; wherein the tissue holder is configured to maintain the in-vitro tissue model in a predetermined spatial relationship relative to the at least one energy emitter.
Example 43. A test method, comprising: providing a tissue sample including tuna meat; placing a tissue treatment device in proximity to the tissue sample; delivering, by the tissue treatment device, ablation energy into the tissue sample to cause a color of the tuna meat to change; and characterizing a performance parameter of the tissue treatment device based on the change in color.
Example 44. The test method of claim 43, wherein characterizing the performance parameter comprises measuring a depth of a lesion formed in the tissue sample, the lesion being defined by an extent of the change in color.
Example 45. The test method of claim 43, wherein the tissue treatment device comprises a cylindrical energy emitter disposed within a lumen of the tissue sample, and wherein characterizing the performance parameter comprises assessing a circumferential uniformity of energy delivery by analyzing a 360-degree profile of the change in color around the lumen.
Example 46. The test method of claim 43, wherein characterizing the performance parameter comprises correlating a variation in an acoustic, optical, or electrical power level delivered by the tissue treatment device with a corresponding variation in a dimension of the change in color.
Example 47. The test method of claim 43, wherein characterizing the performance parameter comprises verifying a non-linear relationship between a biophysical property of the tissue sample and a resulting dimension of the change in color.
Example 48. The test method of claim 43, wherein the tissue treatment device comprises a cylindrical energy emitter disposed within a lumen of the tissue sample, and wherein the characterizing a performance parameter comprises assessing a circumferential uniformity of energy delivery by analyzing a 360-degree profile of the change in color around the lumen. Example 49. The in-vitro tissue model of claim 1, wherein the test article is a radiofrequency ablation device comprising at least one electrode, and wherein the tissue sample is configured to allow direct physical contact between the at least one electrode and a surface of the tissue sample to generate resistive heating therein.
Example 50. The test method of claim 43, wherein the tissue treatment device is a radiofrequency ablation device comprising at least one electrode, and wherein placing the tissue treatment device in proximity to the tissue sample comprises contacting a surface of the tissue sample with the at least one electrode.
Example 51. The in-vitro tissue model of claim 1, wherein the test article is a laser ablation device configured to emit laser energy at a wavelength of approximately 1,064 nm.
Example 52. The test method of claim 43, wherein the ablation energy is laser energy, and wherein characterizing the performance parameter comprises visualizing a subsurface heating effect, wherein a first change in color is observed at a depth within the tissue sample and a second, less significant change in color is observed at a surface of the tissue sample adjacent to the tissue treatment device.
Example 53. The test method of claim 52, wherein visualizing the subsurface heating effect is used to confirm an intimal-sparing performance of the tissue treatment device.
Example 54. The in-vitro tissue model of claim 1, wherein the test article is a microwave ablation device comprising at least one microwave antenna configured to generate volumetric dielectric heating within the tissue sample.
Example 55. The test method of claim 43, wherein the tissue treatment device is a microwave ablation device, and wherein the ablation energy is delivered for a duration of no longer than 60 seconds.
Example 56. The test method of claim 55, wherein the tissue treatment device further comprises an integrated cooling mechanism configured to cool a surface of the tissue sample, and wherein characterizing the performance parameter comprises visualizing a thermal gradient between the cooled surface and a subsurface depth within the tissue sample.
Example 57. The test method of claim 44, wherein characterizing the performance parameter further comprises confirming that the measured depth of the lesion does not exceed a predetermined safety margin.
Example 58. An in-vitro tissue model for use with a test article, comprising: a tissue sample that includes homogeneous tuna meat having an isotropic thermal response; wherein the tuna meat is characterized by a plurality of visually distinct color states, each color state corresponding to a respective temperature to which the tuna meat has been heated within a range of 43° C. to 60° C., thereby providing a visual indication of the thermal effect of the test article within said range.
Example 59. The in-vitro tissue model of example 58, wherein the tissue sample includes a lumen defined therein.
Example 60. The in-vitro tissue model of example 59, wherein the lumen is defined therethrough the tissue sample.
Example 61. The in-vitro tissue model of example 59, wherein the lumen has a diameter in a range of 3 mm to 8 mm.
Example 62. The in-vitro tissue model of example 59, further comprising a porcine renal artery disposed within the lumen.
Example 63. The in-vitro tissue model of example 58, wherein the test article is an ultrasound ablation device.
Example 64. The in-vitro tissue model of example 58, wherein the test article is a radiofrequency ablation device.
Example 65. The in-vitro tissue model of example 58, wherein the test article is a laser ablation device.
Example 66. The in-vitro tissue model of example 58, wherein the test article is a microwave ablation device.
Example 67. The in-vitro tissue model of example 58, wherein the tuna meat has a thermal capacity in a range of 2700 J/Kg/K to 3600 J/Kg/K.
Example 68. The in-vitro tissue model of example 58, wherein the tuna meat has a thermal conductivity in a range of 0.3 W/m*K to 0.7 W/m*K.
Example 69. The in-vitro tissue model of example 59, wherein the tuna meat has a stiffness sufficient to provide a constraining structure that maintains the structural integrity of the lumen when an expandable member of the test article is inflated therein.
Example 70. The in-vitro tissue model of example 58, wherein the tuna meat is from a tuna of a species Thunnus albacares.
Example 71. The in-vitro tissue model of example 58, wherein one of the plurality of visually distinct color states, indicative of thermal lesion formation, corresponds to a temperature in a range of 53° C. to 57° C.
Example 72. An in-vitro test kit for use with a test article having at least one energy emitter, comprising: a tissue holder; and an in-vitro tissue model according to example 58held by the tissue holder; wherein the tissue holder is configured to maintain the in-vitro tissue model in a predetermined spatial relationship relative to the at least one energy emitter.
Example 73. The in-vitro test kit of example 72, wherein the tissue sample of the in-vitro tissue model includes a lumen defined therein.
Example 74. The in-vitro test kit of example 72, further comprising a water bath, wherein the tissue holder is configured to be submerged in the water bath.
Example 75. The in-vitro test kit of example 74, wherein the water bath is configured to be heated to maintain a body temperature.
Example 76. The in-vitro test kit of example 74, wherein the at least one energy emitter is configured to be spaced apart from the in-vitro tissue model by water in the water bath during use.
Example 77. The in-vitro test kit of example 72, further comprising a plurality of temperature sensors configured to be located at respective radial distances from a central axis of the in-vitro tissue model.
Example 78. The in-vitro test kit of example 72, wherein the tissue holder comprises a base and a lid configured to enclose the in-vitro tissue model.
Example 79. The in-vitro test kit of example 78, wherein the lid comprises a plurality of apertures configured to guide the test article and one or more temperature sensors into predetermined positions relative to the in-vitro tissue model.
Example 80. A test method, comprising: providing an in-vitro tissue model comprising a tissue sample that includes homogeneous tuna meat having an isotropic thermal response; placing a tissue treatment device in proximity to the tissue sample; delivering, by the tissue treatment device, ablation energy into the tissue sample to cause a change in color of the tuna meat; and characterizing a performance parameter of the tissue treatment device based on the change in color.
Example 81. The test method of example 80, wherein characterizing the performance parameter comprises measuring a depth of a lesion formed in the tissue sample, the lesion being defined by an extent of the change in color.
Example 82. The test method of example 80, wherein the tissue treatment device comprises a cylindrical energy emitter disposed within a lumen of the tissue sample, and wherein characterizing the performance parameter comprises assessing a circumferential uniformity of energy delivery by analyzing a 360-degree profile of the change in color around the lumen.
Example 83. The test method of example 80, wherein characterizing the performance parameter comprises correlating a variation in an acoustic optical, or electrical power level delivered by the tissue treatment device with a corresponding variation in a dimension of the change in color.
Example 84. The test method of example 80, wherein characterizing the performance parameter comprises verifying a non-linear relationship between a biophysical property of the tissue sample and a resulting dimension of the change in color.
Example 85. The test method of example 80, wherein the tissue treatment device is a catheter comprising an expandable balloon, and wherein placing the tissue treatment device in proximity to the tissue sample comprises inserting a distal portion of the catheter into a lumen defined in the tissue sample and inflating the balloon.
Example 86. The test method of example 80, wherein the tissue treatment device is a radiofrequency ablation device comprising at least one electrode, and wherein placing the tissue treatment device in proximity to the tissue sample comprises contacting a surface of the tissue sample with the at least one electrode.
Example 87. The test method of example 80, wherein the tissue treatment device is a laser ablation device, and wherein characterizing the performance parameter comprises visualizing a subsurface heating effect, wherein a first change in color is observed at a depth within the tissue sample and a second, less significant change in color is observed at a surface of the tissue sample adjacent to the tissue treatment device. In the foregoing specification, the present disclosure has been described with reference to specific exemplary embodiments thereof. It will be evident that various modifications may be made thereto without departing from the broader spirit and scope of the present disclosure as set forth in the following claims. The specification and drawings are, accordingly, to be regarded in an illustrative sense rather than a restrictive sense.

What is claimed is:

1. An in-vitro tissue model for use with a test article, comprising:
a tissue sample that includes homogeneous tuna meat having an isotropic thermal response;
wherein the tuna meat is characterized by a plurality of visually distinct color states, each color state corresponding to a respective temperature to which the tuna meat has been heated within a range of 43°C to 60°C, thereby providing a visual indication of the thermal effect of the test article within said range.

2. The in-vitro tissue model of claim 1, wherein the tissue sample includes a lumen defined therein.

3. The in-vitro tissue model of claim 2, wherein the lumen has a diameter in a range of 3 mm to 8 mm.

4. The in-vitro tissue model of claim 2, further comprising a porcine renal artery disposed within the lumen.

5. The in-vitro tissue model of claim 2, wherein the tuna meat has a stiffness sufficient to provide a constraining structure that maintains the structural integrity of the lumen when an expandable member of the test article is inflated therein.

6. The in-vitro tissue model of claim 1, wherein the test article is an ultrasound ablation device.

7. The in-vitro tissue model of claim 1, wherein the test article is a radiofrequency ablation device.

8. The in-vitro tissue model of claim 1, wherein the test article is a laser ablation device.

9. The in-vitro tissue model of claim 1, wherein the test article is a microwave ablation device.

10. The in-vitro tissue model of claim 1, wherein the tuna meat has a thermal capacity in a range of 2700 J/Kg/K to 3600 J/Kg/K.

11. The in-vitro tissue model of claim 1, wherein the tuna meat has a thermal conductivity in a range of 0.3 W/m*K to 0.7 W/m*K.

12. The in-vitro tissue model of claim 1, wherein the tuna meat is from a tuna of a species Thunnus albacares.

13. The in-vitro tissue model of claim 1, wherein one of the plurality of visually distinct color states, indicative of thermal lesion formation, corresponds to a temperature in a range of 53°C to 57°C.

14. An in-vitro test kit for use with a test article having at least one energy emitter, comprising:
a tissue holder; and
an in-vitro tissue model for use with the test article, the in-vitro tissue model comprising:
a tissue sample that includes homogeneous tuna meat having an isotropic thermal response, wherein the tuna meat is characterized by a plurality of visually distinct color states, each color state corresponding to a respective temperature to which the tuna meat has been heated within a range of 43°C to 60°C, thereby providing a visual indication of the thermal effect of the test article within said range, wherein the in-vitro tissue model is configured to be held by the tissue holder, and wherein the tissue holder is configured to maintain the in-vitro tissue model in a predetermined spatial relationship relative to the at least one energy emitter.

15. The in-vitro test kit of claim 14, further comprising a water bath, wherein the tissue holder is configured to be submerged in the water bath.

16. The in-vitro test kit of claim 15, wherein the water bath is configured to be heated to maintain a body temperature.

17. The in-vitro test kit of claim 15, wherein the at least one energy emitter is configured to be spaced apart from the in-vitro tissue model by water in the water bath during use.

18. The in-vitro test kit of claim 14, further comprising a plurality of temperature sensors configured to be located at respective radial distances from a central axis of the in-vitro tissue model.

19. The in-vitro test kit of claim 14, wherein the tissue holder comprises a base and a lid configured to enclose the in-vitro tissue model.

20. The in-vitro test kit of claim 19, wherein the lid comprises a plurality of apertures configured to guide the test article and one or more temperature sensors into predetermined positions relative to the in-vitro tissue model.

21. A test method, comprising:
providing an in-vitro tissue model comprising a tissue sample that includes homogeneous tuna meat having an isotropic thermal response;
placing a tissue treatment device in proximity to the tissue sample;
delivering, by the tissue treatment device, ablation energy into the tissue sample to cause a change in color of the tuna meat; and
characterizing a performance parameter of the tissue treatment device based on the change in color.

22. The test method of claim 21, wherein characterizing the performance parameter comprises measuring a depth of a lesion formed in the tissue sample, the lesion being defined by an extent of the change in color.

23. The test method of claim 21, wherein the tissue treatment device comprises a cylindrical energy emitter disposed within a lumen of the tissue sample, and wherein characterizing the performance parameter comprises assessing a circumferential uniformity of energy delivery by analyzing a 360-degree profile of the change in color around the lumen.

24. The test method of claim 21, wherein characterizing the performance parameter comprises correlating a variation in at least one of an acoustic, optical, or electrical power level delivered by the tissue treatment device with a corresponding variation in a dimension of the change in color.

25. The test method of claim 21, wherein characterizing the performance parameter comprises verifying a non-linear relationship between a biophysical property of the tissue sample and a resulting dimension of the change in color.

26. The test method of claim 21, wherein the tissue treatment device is a catheter comprising an expandable balloon, and wherein placing the tissue treatment device in proximity to the tissue sample comprises inserting a distal portion of the catheter into a lumen defined in the tissue sample and inflating the balloon.

27. The test method of claim 21, wherein the tissue treatment device is a radiofrequency ablation device comprising at least one electrode, and wherein placing the tissue treatment device in proximity to the tissue sample comprises contacting a surface of the tissue sample with the at least one electrode.

28. The test method of claim 21, wherein the tissue treatment device is a laser ablation device, and wherein characterizing the performance parameter comprises visualizing a subsurface heating effect, wherein a first change in color is observed at a depth within the tissue sample and a second, less significant change in color is observed at a surface of the tissue sample adjacent to the tissue treatment device.

29. The test method of claim 21, wherein the tissue treatment device is a catheter comprising a compliant balloon, the method further comprising disposing a vessel segment within a lumen of the tissue sample, and wherein placing the tissue treatment device comprises inserting a distal portion of the catheter into the vessel segment and inflating the compliant balloon to appose a wall of the vessel segment.

30. The test method of claim 21, wherein the tissue treatment device is a catheter comprising a non-compliant balloon, and wherein placing the tissue treatment device comprises inserting a distal portion of the catheter into a lumen of the tissue sample and inflating the non-compliant balloon into direct contact with an inner surface of the lumen defined by the tuna meat.

31. A medical ablation catheter system, comprising:
an energy emitter configured to deliver ablation energy; and
a controller operably coupled to the energy emitter, the controller having a set of operational parameters stored therein for controlling the delivery of ablation energy;
wherein said set of operational parameters is determined by a method comprising:
(a) delivering ablation energy from a test catheter to an in-vitro tissue model comprising homogeneous tuna meat having an isotropic thermal response;
(b) observing a plurality of visually distinct color states in the tuna meat corresponding to a range of temperatures between 43°C and 60°C; and
(c) selecting the set of operational parameters based on the observed color states to achieve a desired therapeutic effect.

32. The medical ablation catheter system of claim 31, wherein the step of selecting the set of operational parameters comprises selecting parameters operative to form a thermal lesion having a depth that is both sufficient to achieve the desired therapeutic effect and within a predetermined safety margin.

33. The medical ablation catheter system of claim 31, wherein the set of operational parameters comprises at least one of: an energy delivery power, an energy delivery duration, a delivery frequency, or a cooling fluid flow rate.

34. The test method of claim 21, wherein the step of characterizing a performance parameter is performed by an automated imaging system and comprises:
(a) capturing, with an imaging device, image data of the tissue sample after delivery of the ablation energy; and
(b) processing, with one or more processors, the image data to determine the performance parameter based on the change in color.

35. The test method of claim 34, wherein processing the image data comprises quantifying the change in color by representing the color of one or more regions of the tissue sample as numerical values in a defined color space.

36. The test method of claim 35, wherein the defined color space is the CIELAB color space.

37. The test method of claim 34, wherein processing the image data comprises executing an image segmentation algorithm to automatically identify a boundary of a lesion formed in the tissue sample, the lesion being defined by the change in color.

38. The test method of claim 37, further comprising automatically calculating at least one of a maximum lesion depth, a minimum lesion depth, an average lesion depth, or a lesion area based on the identified boundary.
